SEQUENCE LISTING (35 sequences)

SEQ ID NO: 1 - protein, 503 aa, Actinoplanes teichomyceticus
MNSAAQATSTVPELLARQVTRAPDAVAVVDRDRVLTYRELDELAGRLSGRLIGRGVRRGDRVAVLLDRSADLVVTLLAIWKAGAAYVPVDAGYPAPRVAFMVADSGASRMVCSAATRDGVPEGIEAIVVTDEEAFEASAAGARPGDLAYVMYTSGSTGIPKGVAVPHRSVAELAGNPGWAVEPGDAVLMHAPYAFDASLFEIWVPLVSGGRVVIAEPGPVDARRLREAISSGVTRAHLTAGSFRAVAEESPESFAGLREVLTGGDVVPAHAVARVRSACPRVRIRHLYGPTETTLCATWHLLEPGDEIGPVLPIGRPLPGRRAQVLDASLRAVAPGVIGDLYLSGAGLADGYLRRAGLTAERFVADPSAPGARMYRTGDLAQWTADGALLFAGRADDQVKVRGFRIEPAEVEAALTAQPGVHEAVVRAVDGRLVGYVVAEGDAEPAVLRERVGAVLPEYMVPAAVITLDALPLTGNGKVDRAALPAPVFAADAPGREPGTEAE

SEQ ID NO: 2 - protein, 504 aa, Nonomurea sp. ATCC39727
MSAGTRATPTTVLDLFARQVGRAPDAVALVDGDRVLTYRRLDELAGALSGRLIGRGVGRGDRVAVMMDRSADLVVTLLAVWQAGAAYVPVDAALPARRVAFMVADSGACLMVCSEATRDAVPQGVESIALTGEGGCGTSAVTVDPGDLAYVMYTSGSTGTPKGVAVPHRSVAELTGNPGWGVEPGEAVLMHAPYTFDASLFEIWVPLVSGARVVIAAPGAVDARRLREAVAAGVTRVHLTAGSFRAVAEESPESFAHFREVLTGGDVVPAYAVQKVRAACPHVRIRHLYGPTETTLCATWQLLEPGDVVGPVLPIGRPLPGRRAWVLDASLRPVEPGVVGDLYLSGAGLADGYLDRAGLTAERFVADPSAAGRRMYRTGDLAQWTADGELLFAGRADDQVKVRGFRIEPGEVEAALTAQPHVREAVVVAIDGRLIGYVVADGDVDPVLMRRRLAASLPEYMIPAALVTLDALPLTGSGKVDRRALPEPDFASAAPRREPGTEPE

SEQ ID NO: 3 - protein, 500 aa, Streptomyces toyocaensis
MNSVLSTPTVPELFARQAERTPEAVAVVDGDRFVTYRQLDELAGRLAGRLIGRGVRRGDRVAVLMERSADLVVTLLAVWKAGAAYVPVDAAHPAPRVAFVVADSGASLMACSAATAGRVPEGVEPVVVTDEGRGDASAVPVSPGDLAYVMYTSGSTGTPKGVAVPHRSVAELAGNPGWAVKPGDAILMHAPHAFDASLFEIWVPLVSGARVVIAEPGAVDARRLREAIAAGVTKVHLTAGSFRALAEESSESFAGLQEVLTGGDVVPAHAVEKVRKAVPQARIRHLYGPTETTLCATWHLLQPSEALGPVLPIGRPLPGRRAQVLDASLRPLPPGVVGDLYLSGAGLADGYLDRAALTAERFVADPSVPGGRMYRTGDLVQWTADGELLFVGRADDQVKIRGFRIEPGEIEAALTAQPDVHEAVVVAIDGRLIGYAVTDVDPVVLRERLGATLPEYMVPAVVITLDGLPLTRNGKVDRAALPAPVFGTNAAGREPATEAE

SEQ ID NO: 4 - protein, 540 aa, Amycolatopsis orientalis
LPVGRLGVTSEPARASVVERWNSTGEAANRTSVLELFRQQADASPDAVAVMDAARTLSYADLDRESDRLAGYLAAMGVRRGDRVGVVMERGTDLFVALLAVWKAGAAQVPVNVDYPAERIERMLADAGASVAVCLEATRKAVPDGVEPVVMDVPAIDGVRHEAPQVTVGAHDLAYVMYTSGSTGVPKGVAVPHGSVAALASDPGWSQGPDDCVLLHASHAFDASLVEIWVPLVNGSRVMVAEPGAVDAERLREAISRGVTTVHLTAGAFRAVAEESPDSFTGLREILTGGDAVPLASVVRMRRACPDVRVRQLYGPTEITLCATWHVIEPGAETGDTLPIGRPLAGRQAYVLDAFLQPVAPNVTGELYIAGAGLAHGYLGNNGSTSERFIANPFASGERMYRTGDLARWTDQGELLFAGRADSQVKIRGYRVEPGEIEVALTEVPHVAQAVVVAREDHPGDKRLIAYVTAEEGPALAADAVREHLAARMPEFMVPAVVLVLDSFPLTLNGKIDRAALPAPEFTGKAAGREPRTETERVLC

SEQ ID NO: 5 - protein, 539 aa, Amycolatopsis balhimycina
VGRLGVTSEPTRAAVVERWNSTGEAAAETSVLELFRRQAGASPDAVAVVAGERTLSYADLDRESDRLAGHLAGIGVGRGDRVGVVMTRGADLFVALLGVWKAGAAQVPVNVDYPAERIERMLADVGASVAVCVEATRKAVPDGVEPVVVDLPVIGGVRPEAPPVTVGAHDVAYVMYTSGSTGVPKAVAVPHGSVAALASDPGWSQGPGDCVLLHASHAFDASLVEIWVPLVSGARVLVAEPGTVDAERLREAVSRGVTTVHLTAGAFRAVAEESPDSFIGLREILTGGDAVPLASVVRMRQACPDVRVRQLYGPTEITLCATWLVLEPGAATGDVLPIGRPLAGRQAYVLDAFLQPVAPNVTGELYLAGAGLAHGYLGNTAATSERFVANPFSGGGRMYRTGDLARWTDQGELVFAGRADSQVKIRGYRVEPGEVEVALTEVPHVAQAVVVAREGQPGEKRLIAYVTAEAGSALESAAVRAHLATRLPEFMVPSVVVVLESFPLTLNGKIDRAALPAPEFAGKAAGREPRTEAERVLCG

SEQ ID NO: 6 - protein, 539 aa, uncultured soil bacterium
STVADVDVTSAAERALVVDEWGAAAEAAPSRLALELFDGQVESRRDAIAVVDRDQAMSYGVLAEDAERLAGYLNGRGVRRGDRVAVVVERSHDLIATLLAVWKAGAAYVPVDPAYPLERVKFMLADADPAAVVCTAGYRDSVLDGGLDPIVLDDPQTRQAVSECSRLSVGTTADDVAYVMYTSGSTGTPKGVAVSHGNVAALVGEPGWRVGPDDAVLMHASHAFDISLFEMWVPLVSGARVVLAGSGAVDGAALAAYVADGVTAAHLTAGAFRVLAEESPESVAGLREVLTGGDAVPLAAVERVRRTCPDVRVRHLYGPTEATLCATWLLLEPGDETGPVLPIGRPLAGRRVYVLDGFLRPVPPGVAGELYVAGAGVAQGYLERPALTAERFVADPFVAHGRMYRTGDLAYWTGKGALAFAGRADDQVKIRGYRVEPGEIEVVLAGLPGVGQAVVLARDEHLIGYAVAEAGHELDPVRLREQLADTLPEFMVPAAVLVLGELPLTVNGKVDRQALPGPDFASKAAGRAPATDAERVLCG

SEQ ID NO: 7 - protein, 535 aa, Actinoplanes teichomyceticus
LTVAAIDVTSAAERDRVARWGAAVGARPDRLALDLFARQVAQRPDEVAVADGDRVMSFGELAERADRLAGHLSARGVRRGDRVAVVMERSGELIATLLAVWRAGAAFVPVDPAYPAERVKFLLTDAEPVAAVCTAAFRAAVLDGGLEAIVVDDPGTWPAVAPCPPVPTGPDDLAYVMYTSGSTGTPKGVAVSHGDVAALVGDPGWRTGPGDTVLMHASHAFDISLFEIWVPLLSGARVMIAGPGAVDGAALAAQVAAGVTAAHLTAGAFRVLAEESPESVAGLREVLTGGDAVPLAAVERVRRACPDVRVRHLYGPTETTLCATWWLLEPGDETGPVLPIGRPLAGRRVYVLDAFLRPLPPGTTGELYVAGAGVAQGYLGRPALTAERFVADPFAPGGRMYRTGDLAYWTEQGTLAFAGRADDQVKIRGYRVEPGEVEAVLGGLPGVAQAVVCVRGEHLIGYVVAEAGRDLDPERLRARLAATLPEFMVPAAVLVLADLPLTVNGKVDRPALPEPDFAAKSTGRAPATAAERILC

SEQ ID NO: 8 - protein, 538 aa, Streptomyces toyocaensis
LPVGRLGVTSDATRTSEVERWNATGEAAGGASVVELFRRRSAGTPDAVAVVDGDRTLSYGDLDRESDRLAGRLAETGVRRGDHVGVVLERGADLFVAFLAVWKAGAAYVPVHVDYPPVRIERMLADAGVTVAVCAEGTRNAVPDGLEPVPVDAPWAGETRHETPTVTARDAAYVMYTSGSTGEPKGIVVPHGSVAALAGDPGWALDADDCVLMHASHAFDASLFEIWAPLVRGARVMVAEPGAVDTQRLREAVARGVTTVHLTAGSFRVLAEESPGSFDGLREILTGGDVVPLASVAQLRRACPDVRVRHLYGPTETTLCGTWHLLEPGDEPGDVLPIGRPLAGRRAYVLDAFLQPVAPNVTGELYLAGVGLALGYLGARGATSERFVADPFVPGERMYRTGDLARRNDRGELLFAGRADAQVKIRGYRVEPTEIETVLAEAPQVAQTVVVAREDGPGEKRLIAYAIAEPDQVLDPEALREHLAARLPEFMVPAAVVVLDDFPLTINGKIDREALPAPEFSAKPAGREPRTEAERVLC

SEQ ID NO: 9 - protein, 522 aa, Streptomyces lavendulae
VLVGRVGLVGRLERGLVVEGWNATAGDVPSGSSVLEMFRARVAQAPEAVAVVDGERQVSYGELDADSNRMAAYLQGRGVGRGDRVAVRLERSIDLIAALLGVWKAGAAYVPVDSAYPAERVAFMVEDSAPVLTIDDPSVVTAEGEPEVVETAGGDIAYVMYTSGSTGTPKGVAVPHASVAALVGEPGWGVGPGDAVLFHAPHAFDISLFEVWVPLASGGRIVVAEPSMAVDGAAVRRHIADGVTHVHVTAGLFRVLAEEASDCFDGVHEVLTGGDVVPLEAVERVRAACPDVRVRHLYGPTEVSLCATWHLFEPGEEQGEVLPLGRPLNNRQVYVLDPFLQPVPPGVTGELYVAGAGLARGYLGRAGLSAERFVASPFADGERMYRTGDLVRWTTGVELVFVGRADAQVKIRGFRVELGEVEAALAAQPAVAQAVVVAREDRPGEKRLVGYLVPSGEEPDTEAVHASLADRLPEYMVPAALVVLDALPLTVNGKVDHKALPAPEFTATASREPRTAAEKLLC

SEQ ID NO: 10 - DNA, 1742 bp, Artificial Sequence
  source 1..1742: /organism=“Artificial Sequence” /note=“Synthetic DNA” /mol_type=“unassigned DNA”
catatgagcgctggcactagagcaaccccgaccaccgtactcgacctgttcgcccgtcaggtcggccgtgcgccggacgctgtcgcgctggtggacggtgaccgtgtcctgacctaccgccgtctggatgagctggcgggtgcattgagcggccgtctgattggtcgtggtgtcggccgtggcgatcgcgtggccgtcatgatggaccgcagcgcggatctggtcgttaccctgctggcagtttggcaggcaggtgcggcgtacgttccggtggacgcagcactgcctgcgcgtcgtgtggccttcatggtggcggatagcggtgcgtgtctgatggtgtgctctgaggcgacccgcgatgccgtgccgcaaggtgttgagagcatcgcactgaccggcgaaggtggttgtggtactagcgcggtcacggtggacccaggcgacctggcctatgtgatgtacacttccggctctaccggcaccccgaagggtgtggctgtccctcaccgctcggtggcagagctgaccggtaatccgggttggggtgtggagcctggtgaggcggttctgatgcacgcgccgtacacgtttgatgcaagcttgtttgagatttgggttccgctggtgagcggtgcgcgtgttgtgattgctgctccgggtgcggtcgacgcccgtcgcttgcgtgaagcggtcgcagctggcgtgacccgcgttcatttgacggcgggtagctttcgtgccgtggccgaagagagcccggagagcttcgcgcacttccgcgaagttctgaccggtggcgatgtggtgccggcctatgctgtccagaaagttcgtgccgcgtgtccacatgttcgtatccgccatttgtatggtccgaccgaaacgacgctgtgcgctacctggcagctgctggaaccgggcgacgtggttggcccggttctgccgatcggtcgcccgctgccgggtcgtcgcgcatgggttctggatgcgagcctgcgtccggtcgagccaggcgtcgtcggcgacctgtacctgtccggtgcaggcctggcggacggttatctggaccgtgccggtctgacggcggaacgtttcgttgccgatccaagcgctgccggtcgtcgcatgtatcgcaccggtgacctggcgcagtggaccgcggacggcgagctgctgtttgcaggccgtgccgatgatcaagtgaaggttcgtggcttccgtattgagccgggtgaggttgaggcagcgctgaccgcgcagccgcacgtccgcgaagcggtggttgttgcgatcgacggtcgcctgatcggctacgtcgtggccgatggtgacgtggatccggtcctgatgcgtcgccgcctggcggcaagcctgccggaatacatgattcctgcggcactggtgaccttggacgcactgccgctgacgggcagcggtaaggttgaccgccgtgcgttgccggagccggattttgcgagcgctgcccctcgtcgtgaaccgggcacggaaccggaggacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctcggttctcgtagccaccatcatcaccatcactgacctgcagg

SEQ ID NO: 11 - DNA, 1730 bp, Artificial Sequence
  source 1..1730: /organism=“Artificial Sequence” /note=“Synthetic DNA” /mol_type=“unassigned DNA”
catatgaactccgtactgtccaccccaaccgtcccagagctgttcgcgcgtcaggcagagcgcactccggaagctgtggcagtcgttgatggtgatcgctttgtcacctaccgtcaactggacgagctggcaggccgtctggcaggccgcttgattggtcgtggtgttcgtcgcggcgaccgtgtcgcggtcctgatggaacgttctgcggatctggtcgtcaccctgctggccgtttggaaggctggcgctgcgtacgttccggttgatgcggcgcatccggcaccgcgtgtggcattcgtggtggctgacagcggtgcgagcctgatggcatgctcggcagcgacggccggtcgcgtgccggagggcgttgagccagtggtcgtgactgatgaaggtcgtggcgacgcgagcgcggttccggtcagcccgggtgatctggcctacgtgatgtataccagcggcagcacgggcacgccgaaaggtgtcgctgttccgcatcgcagcgttgcggagctggcgggtaatccaggttgggcggttaaaccgggcgatgcgattctgatgcacgcgcctcacgcgtttgacgccagcctgttcgagatctgggttccgttggttagcggtgcccgcgttgtcatcgcggagccaggcgctgttgatgcccgtcgtctgcgcgaagcgatcgcagcaggtgttaccaaagttcacctgactgccggtagctttcgtgctctggccgaagagagcagcgaaagctttgccggcctgcaggaagtgctgacgggtggcgatgtggtgccggctcacgcagtcgaaaaggtccgtaaggcagtgccgcaagcgcgcattcgtcacctgtatggcccgaccgaaaccacgctgtgtgccacctggcatctgctgcagccgagcgaggcgttgggtccggtgctgccgattggccgtccgttgccgggtcgtcgtgcccaagtgctggacgcaagcctgcgtccgctgccgcctggcgtggtgggcgatctgtatttgagcggtgcgggcctggcggacggttacctggatcgtgcggccttgaccgcagagcgcttcgtggccgatccgtccgttccgggtggccgtatgtaccgcacgggtgacctggtccagtggacggctgacggtgagctgctgtttgttggtcgtgcggacgaccaggtgaagatccgtggtttccgtatcgaaccgggtgaaatcgaagcagcactgacggcgcaaccggacgttcatgaggcggttgtggtcgcgatcgacggtcgcctgattggttatgcagtgaccgacgtggatccggtggttttgcgcgagcgtttgggcgcgaccctgccggaatacatggttcctgcagtcgttatcaccttggatggcctgccgctgacccgtaatggcaaagtcgaccgtgcggcgctgccggcaccggtttttggcaccaacgccgcaggtcgcgagccggcgaccgaggcggaggacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctcggttctcgtagccaccatcatcaccatcactgacctgcagg

SEQ ID NO: 12 - DNA, 1667 bp, Artificial Sequence
  source 1..1667: /organism=“Artificial Sequence” /note=“Synthetic DNA” /mol_type=“unassigned DNA”
catatgctgccggtcggccgcttaggcgttacttcagaacctgcgagagccagcgttgttgagcgctggaatagcaccggcgaagcggcgaatcgcaccagcgttttggagctgttccgtcaacaagctgatgcgtccccggacgcggtggccgtgatggatgcggctcgcacgctgtcgtatgctgacctggatcgcgagagcgaccgtctggcaggttacctggcggcaatgggtgtccgccgtggtgatcgtgtcggtgttgttatggagcgtggtacggatctgttcgttgctctgctggcagtgtggaaagcaggcgcagcacaggtcccggttaacgttgattatccggcggagcgtattgagcgtatgctggcggatgcgggtgcgagcgttgcggtgtgtctggaagccacccgtaaagcagtgccggatggtgtggagccggttgtcatggacgtcccggccatcgacggcgtccgccatgaggctccgcaggtgacggttggtgcacacgacctggcctacgtcatgtatacgagcggcagcacgggcgtgccgaagggtgtcgccgtgccgcatggctctgttgcggccctggcgagcgaccctggttggtcccaaggcccggacgactgcgtcctgctgcacgcaagccacgcctttgatgcttccttggtcgaaatctgggtcccgctggtcaatggtagccgcgtcatggttgcggaaccgggtgcggtggatgcggaacgtttgcgtgaagcgatcagccgtggtgtgacgaccgttcacctgacggcgggtgcattccgtgcagtcgcagaggagagcccggactccttcaccggcctgcgcgagatcctgaccggcggtgatgcggttccgttggcaagcgtcgttcgtatgcgtcgtgcttgcccggatgtacgtgttcgtcagttgtacggtccgaccgaaattaccctgtgtgcaacctggcacgtgattgagccgggtgccgaaacgggtgacaccctgccgattggtcgcccgctggcaggccgtcaggcgtatgtgctggatgcgtttctgcaaccagttgcacctaacgtgacgggcgaattgtacattgctggtgcgggcctggcacatggctatctgggcaacaacggtagcaccagcgaacgttttatcgcgaacccgttcgcgtctggcgaacgcatgtaccgtaccggcgatttggcacgttggaccgaccagggtgaactgctgttcgccggtcgcgctgacagccaagtgaaaattcgcggttaccgcgttgagccaggcgagatcgaagtggcactgacggaggtgccgcacgttgcccaggcggtcgtggtggcccgtgaggaccatccgggtgacaagcgcctgatcgcctacgttactgccgaggaaggtccggcgctggcggcagatgcggtacgtgagcatctggcagcgcgtatgccggagtttatggttccggcggtggtgctggtgctggatagcttcccactgaccctgaatggtaagattgaccgtgcggcgctgccggcaccagaatttaccggcaaagcagcgggtcgtgagccgcgcaccgagactgagcgtgtcttgtgcggtagccgttcccaccaccatcatcaccactaacctgcagg

SEQ ID NO: 13 - DNA, 1661 bp, Artificial Sequence
  source 1..1661: /organism=“Artificial Sequence” /note=“Synthetic DNA” /mol_type=“unassigned DNA”
catatggtaggcagactgggcgtgacgagcgaaccgacgagagcagcggtggtggagcgttggaactcgaccggcgaggcggctgccgaaacgagcgtgctggaactgtttcgtcgccaggcaggtgcgagcccggatgcagttgccgtcgtggcgggtgaacgtacgctgagctacgcggatctggaccgtgagagcgatcgtctggcgggtcatttggcaggcattggcgttggtcgtggtgatcgcgtcggtgtagtgatgacccgtggtgcggacttgtttgtcgcactgctgggcgtttggaaagccggtgcagcacaagtgcctgttaacgttgattacccggctgagcgtatcgaacgtatgctggctgatgtcggtgcaagcgtcgcggtgtgtgtagaggcgacccgcaaagcagtgccggatggtgttgagccggtcgttgtcgatctgccggttatcggtggtgttcgtccggaagccccacctgtgacggtgggtgcccacgacgtcgcgtacgtcatgtacacgagcggctccacgggcgttccgaaggcagtggcggtcccacacggttctgtggcggcactggcaagcgacccgggttggagccagggtccgggtgactgcgttctgctgcacgcatctcatgcgtttgacgcatctctggtggagatttgggttccgctggtgagcggtgcccgcgttctggtggcggagccgggcacggtggatgcggaacgcctgcgtgaagcggttagccgcggtgtcaccaccgtgcacctgaccgcaggtgccttccgtgcggttgccgaagagagcccagatagcttcatcggtctgcgtgagatcctgacgggtggtgacgccgtcccgctggcgagcgtggttcgcatgcgccaagcgtgcccggacgttcgtgtccgtcagctgtatggcccgaccgagatcaccctgtgcgccacctggctggtcctggaaccgggtgcggcgactggtgacgtcctgccgattggccgtccgctggcaggtcgccaagcctatgtgttggatgctttcctgcaacctgttgcgccgaacgtcaccggcgaactgtacctggcgggtgcaggcctggctcacggttatctgggtaatactgccgcgaccagcgagcgcttcgttgcgaacccgttttccggcggtggccgtatgtatcgtacgggtgacctggcacgctggaccgaccagggcgagctggtgttcgctggccgtgcggatagccaggttaagatccgtggttaccgtgtcgaaccgggcgaagttgaggtcgcactgaccgaggtgccgcatgttgcgcaggcagtcgtggtggcccgtgagggccaaccgggtgagaaacgcctgattgcgtatgtgaccgcggaagcgggttccgcgttggaatctgcggcggttcgcgcccacctggccacccgtctgccggagttcatggtcccgagcgtcgtggtcgttttggagtccttcccgttgaccctgaatggcaagattgaccgtgccgctttgccagcgccggaatttgcgggtaaagcagcgggtcgtgagccgcgtaccgaagcagagcgtgttttgtgtggtagccgcagccatcatcatcaccaccactaacctgcagg

SEQ ID NO: 14 - DNA, 1661 bp, Artificial Sequence
  source 1..1661: /organism=“Artificial Sequence” /note=“Synthetic DNA” /mol_type=“unassigned DNA”
catatgagcacggtagcagacgtagacgtaaccagcgcagcagaacgcgcgctggtagtggatgaatggggtgcagcggcggaggcggcaccgagccgcctggcactggaactgttcgacggccaagtggagagccgtcgcgatgccatcgcggtcgttgaccgcgaccaggccatgagctatggcgttctggcggaggatgccgagcgtctggccggctatttgaatggtcgtggcgttcgtcgcggtgatcgtgtcgcggttgttgtggagcgctctcatgacctgattgccaccctgctggcggtctggaaggcaggcgcagcctatgtcccggtagatccggcatacccgctggaacgtgtcaagttcatgctggcagacgcggacccggcagctgtcgtctgtaccgcaggctatcgtgacagcgtcctggacggtggcttggaccctatcgttttggatgatccgcaaacccgtcaggcggtcagcgaatgttctcgtttgtccgtgggcaccaccgccgacgacgttgcgtatgtcatgtacacgagcggtagcaccggcaccccgaaaggcgtcgccgtcagccacggtaacgttgcagcgctggtgggtgagccgggttggcgtgttggcccggatgacgcagttctgatgcacgcaagccacgccttcgacatcagcctgtttgaaatgtgggttcctctggtgtccggtgctcgcgtggtgctggctggttccggtgcggtggacggtgcggcgctggcggcgtatgtggctgatggcgtgaccgcagcgcatctgacggcaggcgctttccgtgttctggctgaggagagcccggagtccgttgcgggtctgcgtgaagttttgaccggcggtgatgcggttccactggcagcggttgaacgtgttcgtcgtacctgcccggacgtgcgcgtgcgtcacctgtacggcccgacggaggcaaccctgtgcgcgacgtggctgctgttggaaccgggcgatgaaacgggtccggttttgccaatcggccgtccgctggcgggtcgccgcgtctacgtgctggatggtttcctgcgtccggttccaccgggtgtggctggtgagctgtacgtagccggtgcaggtgtcgctcaaggctacctggaacgtccggcgttgactgcggagcgttttgtcgccgatccgtttgtggcccacggccgtatgtaccgtactggtgatctggcgtactggacgggtaaaggtgctctggcatttgcgggtcgtgcagatgatcaggtgaaaattcgtggctaccgcgtggagccgggtgaaattgaggtggttctggccggtctgccgggtgttggccaggcggtcgtgctggcccgtgatgaacacctgattggctatgcagtggctgaggctggtcatgagctggacccggtgcgcctgcgtgagcagctggcggacaccctgccggagttcatggtcccggcagcggtcctggttttgggcgaactgccgctgacggtcaacggtaaggttgatcgccaagcgttgccaggtccagactttgcaagcaaagcagcgggtcgcgctccggcgaccgacgcggagcgcgtgctgtgcggttctcgtagccaccatcatcaccatcactaacctgcagg

SEQ ID NO: 15 - DNA, 1652 bp, Artificial Sequence
  source 1..1652: /organism=“Artificial Sequence” /note=“Synthetic DNA” /mol_type=“unassigned DNA”
catatgctcaccgtagccgccatcgacgtcacctcagccgccgaacgcgaccgtgtcgcgcgttggggtgcggctgtcggtgctcgcccggaccgtctggcgctggacctgttcgcccgtcaagttgctcaacgcccggacgaggtggccgttgcagacggcgaccgcgtcatgagcttcggcgaactggcggagcgtgcggatcgtttggcgggtcatctgagcgcacgcggcgttcgtcgtggcgatcgcgtggcggttgtcatggagcgctcgggcgaactgattgcgaccctgctggcggtgtggcgcgcaggcgcagcgtttgtgccggttgatccggcataccctgcggagcgcgttaagtttttgctgaccgacgctgagccggtggcggcagtgtgcaccgctgcatttcgtgcggcggtcctggatggcggtctggaggccattgtcgtagatgatccgggtacgtggccggctgtcgcgccgtgtcctccggtgccgactggtccagatgacctggcatacgtgatgtataccagcggctccacgggcaccccgaaaggtgtggctgttagccacggtgatgttgcggcgttggttggcgatccgggctggcgcacgggtccgggtgacaccgtgctgatgcacgcttctcacgcattcgacatttccttgttcgaaatctgggtcccgctgctgagcggtgcgcgtgtgatgatcgccggtccaggtgcagtcgatggtgccgcgctggccgctcaggttgcagcaggtgtcaccgctgcgcatctgaccgctggcgcattccgtgttctggcggaagaaagcccggagagcgtcgcgggtctgcgtgaggtgctgacgggtggcgacgcagttccgctggcagcagtggagcgcgtgcgccgtgcctgcccggacgttcgtgttcgtcacctgtatggcccgaccgaaaccacgctgtgtgcaacgtggtggttgctggaaccgggtgatgaaacgggtccagtgctgccgatcggtcgtccgctggccggtcgccgcgtgtatgtgctggacgcattcctgcgtccgctgccgccaggcaccaccggcgagctgtatgttgcgggtgcgggtgttgcacagggctacttgggtcgtccggcgctgacggcggaacgctttgttgcggacccttttgcgcctggtggccgtatgtaccgcactggtgatttggcctactggaccgagcagggtactctggcgtttgcgggtcgtgcggacgatcaagtgaaaattcgtggttatcgtgttgagccgggtgaagtggaggcggtgctgggcggcttgccgggtgtcgcacaggccgtagtatgcgtccgtggtgagcatctgattggttacgtggttgccgaagccggtcgcgatctggacccggagcgtctgcgtgcgcgtttggcagccaccctgccggagttcatggtgccagcggctgtgctggtcctggcagatttgccgctgacggttaacggtaaggtcgatcgtccggctctgccggaaccggacttcgccgctaaaagcacgggccgtgcaccggccacggctgcggaacgcatcctgtgtggcagccgtagccatcaccaccaccatcactaacctgcagg

SEQ ID NO: 16 - DNA, 1661 bp, Artificial Sequence
  source 1..1661: /organism=“Artificial Sequence” /note=“Synthetic DNA” /mol_type=“unassigned DNA”
catatgcttccagtcggtcgcttaggcgtaacctcagatgcaactcgcacgagcgaagtcgagcgctggaatgcgacgggcgaagctgcgggtggtgcgagcgtggttgagctgttccgtcgtcgttctgcgggcaccccggatgccgttgcggtcgtggacggtgatcgcaccctgagctacggcgacctggaccgcgagtcggatcgtctggccggtcgcttggcagaaacgggtgtgcgtcgcggcgatcacgtgggtgtcgtcctggaacgcggtgcggacctgttcgtagccttcctggcggtttggaaggcgggtgctgcttacgttccagttcacgtggattatccgccggtccgtattgaacgtatgctggcggatgccggtgtgacggtcgcggtttgtgcggaaggtacgcgcaacgccgtgccggacggcctggagccggttccggttgatgcaccgtgggcgggtgaaacccgccacgaaaccccgacggtgacggctcgtgacgcggcctacgttatgtacaccagcggcagcaccggcgagccgaaaggcatcgttgttccgcatggcagcgttgccgcactggcaggtgacccaggttgggctctggacgctgacgattgcgtgctgatgcacgcgagccatgcgttcgatgcttccttgtttgaaatttgggcaccgctggtccgtggcgcacgtgtcatggtcgcggagcctggtgcggtggatacccagcgtctgcgtgaagcggtggcgcgtggtgtcaccaccgtgcacctgaccgccggtagcttccgcgtcctggcggaggagtctccgggttcttttgatggtctgcgcgagatcctgactggtggcgacgtggtgccgctggcaagcgtcgcacaattgcgtcgcgcctgcccggatgtgcgcgtccgtcacctgtatggcccgacggaaaccaccctgtgcggcacctggcacctgctggagcctggcgacgaaccgggtgacgtgctgccgatcggtcgtccgctggcaggccgtcgtgcgtatgtgctggacgcatttctgcaaccagtggcgccgaatgttactggcgagctgtatctggcgggtgtgggtttggcgctgggttacttgggtgcccgtggtgcgaccagcgagcgttttgttgcagacccgttcgttcctggtgagcgtatgtaccgtactggcgatctggcgcgtcgcaacgatcgcggtgaattgctgtttgcaggccgtgcagacgcgcaggttaagattcgtggttatcgtgtcgagccgacggagatcgaaaccgtattggcagaagcaccgcaagtggcacagacggtcgttgttgcccgcgaggacggtccgggtgagaagcgtctgattgcatacgcgattgcggaaccggaccaggttctggacccggaggccttgcgtgaacatctggcagcgcgtttgccggagtttatggttccggcagctgtggttgtgctggatgacttcccgctgaccatcaacggcaaaatcgaccgtgaagcgctgccggcaccggagttcagcgcaaaacctgctggccgtgagccgcgtaccgaggcggagcgtgttctgtgtggttcccgcagccatcatcaccaccaccattaacctgcagg

SEQ ID NO: 17 - DNA, 1613 bp, Artificial Sequence
  source 1..1613: /organism=“Artificial Sequence” /note=“Synthetic DNA” /mol_type=“unassigned DNA”
catatggtactggtaggcagagtaggcttagtgggcagactcgaacgtggtctggtcgtcgaaggttggaacgccaccgccggtgacgtgccgtctggcagctctgtcttggagatgtttcgtgcgcgtgtggcgcaagcaccggaggccgttgcggttgttgacggtgagcgccaggtgagctacggcgagctggacgcggacagcaaccgtatggcagcgtacctgcaaggtcgtggtgtgggtcgtggcgaccgcgttgcagtccgcttggagcgttctatcgacctgattgcagcgttgttgggtgtgtggaaggcgggtgccgcgtatgtgccggtggatagcgcgtatccggccgaacgtgtggcgttcatggtcgaagatagcgcaccagtgctgacgatcgatgatccgtcggttgtcaccgcagagggtgagccggaggtcgtggaaaccgcaggtggtgacattgcttacgtgatgtacacgagcggcagcaccggcacgccgaaaggcgtggccgttccgcacgcatcggtggccgcgttggtcggtgaaccaggttggggtgttggtccgggtgacgcagtgctgttccatgcgccacacgcctttgacatctctctgtttgaagtttgggtcccgctggcgagcggtggccgtatcgttgtcgcagagccgagcatggcggtggacggtgcggccgttcgtcgtcatatcgcggacggtgtgacccacgtccacgtaacggcgggtctgttccgtgtgctggcagaagaggcaagcgattgtttcgatggtgtccatgaggtcctgactggtggtgacgtcgttccgctggaagcggtggagcgcgttcgcgctgcgtgcccagatgtgcgcgttcgccacctgtatggcccgactgaggtttctttgtgcgctacctggcacttgttcgaaccgggtgaagaacagggcgaggtcctgccgctgggtcgtccgctgaacaatcgtcaagtttatgttctggacccgtttctgcaaccggttcctccgggcgttacgggtgagctgtacgttgcgggtgcaggtctggcgcgtggctacctgggtcgtgccggtctgtcggcggaacgcttcgtggcatccccgtttgcagacggcgaacgtatgtatcgtaccggcgacctggtgcgttggaccactggtgtcgagctggtgttcgtgggtcgcgcagacgcgcaagtgaaaattcgtggtttccgcgttgagttgggtgaggtcgaagcggcactggctgcccagcctgcggtggcccaggcagtggttgttgcgcgcgaggaccgtccgggcgagaagcgtctggtgggctacctggtgccatctggtgaagaaccggacactgaagcagttcacgcaagcctggcagatcgtttgccggaatacatggttccggctgcgctggtggtgctggacgcgctgccgctgacggttaatggtaaggtggaccataaggcgctgccggccccggaatttaccgcaacggccagccgtgaaccgcgtactgccgctgaaaagctgctgtgcggcagccgtagccaccaccatcatcaccactaacctgcagg

SEQ ID NO: 18 - protein, 69 aa, Actinoplanes teichomyceticus
MTNPFDNEDGSFLVLVNGEGQHSLWPAFAEVPDGWTGVHGPASRQDCLGYVEQNWTDLRPKSLISQISD

SEQ ID NO: 19 - protein, 69 aa, Actinoplanes teichomyceticus
MTNPFDNEDGSFLVLVNGEGQHSLWPAFAEVPDGWTGVHGPASRQDCLGYVEQNWTDLRPRSLVEQADA

SEQ ID NO: 20 - protein, 69 aa, uncultured soil bacterium
MTNPFDNEDGTFFVLVNDEGQHSLWPTFAEVPAGWTRVHGEATRQECLAYVEENWTDLRPKSLIQALGA

SEQ ID NO: 21 - DNA, 238 bp, Artificial Sequence
  source 1..238: /organism=“Artificial Sequence” /note=“Synthetic DNA” /mol_type=“unassigned DNA”
ggatccaggaggaattacatatgaccaatccgttcgacaacgaggacggttccttcctcgtgctcgtcaacggcgagggccagcattcgctgtggccggctttcgccgaggtcccggacggctggacgggggtccacggtccggcctcccggcaggattgtctcggctacgtcgagcagaactggacggacctgcggcccaagagtctgatctcgcagatcagcgactgacctgcagg

SEQ ID NO: 22 - DNA, 238 bp, Artificial Sequence
  source 1..238: /organism=“Artificial Sequence” /note=“Synthetic DNA” /mol_type=“unassigned DNA”
ggatccaggaggaattacatatgaccaatccgttcgacaacgaggacggttccttcctcgtgctcgtcaacggcgagggccagcattcgctgtggccggctttcgccgaggtcccggacggctggacgggggtccacggtccggcctcccggcaggattgtctcggctacgtcgagcagaactggacggacctgcggcccaggagcctggtcgagcaggccgacgcgtgacctgcagg

SEQ ID NO: 23 - DNA, 238 bp, Artificial Sequence
  source 1..238: /organism=“Artificial Sequence” /note=“Synthetic DNA” /mol_type=“unassigned DNA”
ggatccaggaggaattacatatgaccaacccgttcgacaacgaggacggcaccttcttcgtgctggtcaacgacgagggccagcactccctctggccgaccttcgccgaggtgcctgccggctggacccgcgtgcacggtgaagccacccggcaggagtgcctcgcgtatgtcgaggagaactggacggacctgcggccgaagagcctcatccaggccctcggcgcctgacctgcagg

SEQ ID NO: 24 - DNA, 1742 bp, Artificial Sequence
  source 1..1742: /organism=“Artificial Sequence” /note=“Synthetic DNA” /mol_type=“unassigned DNA”
catatgaactccgcagcgcaggccacatcgacggtgccggagctgctcgcccggcaggtgacccgggcccccgatgcggtggccgtggtggaccgggaccgggttctgacgtaccgggaactcgatgagctcgcgggccggttgtccggacgtctgatcggccggggcgtccgccgcggggaccgcgtggcggtcctgctggaccgttcggcggacctggtggtgacgctgctcgcgatctggaaggccggggcggcgtatgtgccggtcgatgccggctatcccgcgccgcgtgtggcgttcatggtggcggactcgggagcctcccgcatggtgtgctcggccgcgacgcgtgacggcgtaccggaggggatcgaggcgatcgtcgtcacggatgaggaggcgttcgaggcctcggcggccggggcgcgaccgggagatctggcgtacgtgatgtacacctccggctcgaccggcatcccgaagggcgtggcggttccgcatcgcagcgtcgcggagctggccgggaatcccggctgggcggtggagccgggcgacgcggtcctgatgcacgcgccgtacgccttcgacgcgtcgctgttcgagatctgggtgccgctggtttccgggggccgggtggtgatcgccgagccggggccggtggacgcccggcgcctgcgggaggcgatcagctccggggtgaccagggcgcatctgaccgccggcagcttccgcgcggtggcggaggagtcgccggagtccttcgccgggctgcgcgaggtgctgaccggcggtgacgtggtgccggcacacgccgtggcgcgggtccgctcggcctgtccccgggtgcggatccggcacctgtacggcccgacggagacgacgctgtgcgccacatggcatcttctggagccgggggacgagatcggcccggtgttgccgatcggccgtccgctcccgggccggcgcgctcaggtgctcgacgcgtcgctgcgggccgtggcgccgggcgtgatcggtgacctgtacctgtccggcgccggtctggctgacggctacctgcgccgggcagggctgacagcggagcgattcgtggccgacccgtccgcgcccggggcgaggatgtaccgcaccggcgacctcgcgcagtggaccgccgacggtgcgttgctgttcgcgggccgggccgacgaccaggtgaaggttcgcggcttccggatcgagccggccgaggtcgaggccgcgttgaccgcgcagccgggcgtccacgaggccgtggtccgagcggtcgacgggcgcctggtcggctatgtggtggcggagggggacgcggaaccggctgtcctgcgcgagcgtgtcggtgcggtgctgccggagtacatggtcccggccgcggtgatcacactggacgcgctgccgctgaccggcaacggcaaggtggaccgggcggctctgccggctccggtcttcgcggcggacgctccggggcgcgaacccggcaccgaggcggagcgcgtgctgtgcgggctgctgtccgaggtgctcggcctgaaccgggtcggagtcgacgagagcttcttcgagctgggcggagactccatcgcggcgatccggctggcggcgcgtgcgtcccgggcgggcctgctcgtgacgcccgcccagatcttcaaggagaggactgtcgcacggctggcggccgtgggttctcgtagccaccatcatcaccatcactgacctgcagg

SEQ ID NO: 25 - DNA, 3164 bp, Artificial Sequence
  source 1..3164: /organism=“Artificial Sequence” /note=“Synthetic DNA” /mol_type=“unassigned DNA”
catatgagcaccgttgcggatgtggacgttaccagcgcagccgaacgtgccctggtagtggatgagtggggcgctgctgcggaggcagcgcctagccgcctggcactggagctgtttgacggtcaagtggagagccgtcgtgacgcgattgcggtcgttgatcgtgatcaggcaatgagctacggcgttctggccgaagatgcagaacgcctggcgggctacctgaatggccgtggtgttcgtcgtggtgatcgcgtggcagtcgtggtggaacgtagccatgacttgattgccactctgttggccgtttggaaagctggcgcagcctacgtgccggttgacccggcatacccgctggagcgcgtgaaattcatgctggccgatgccgacccggcagcagtggtttgtacggcaggctatcgtgactccgtcttggatggtggtttggacccgattgttctggacgatccgcagacgcgccaagcagtcagcgaatgcagccgtctgtctgtaggcactactgcggacgatgtagcttacgtcatgtacacctctggttcgaccggcaccccgaaaggcgtcgcagtttcccacggcaacgtcgcggctctggttggcgaaccgggctggcgcgtcggtccggacgatgccgttctgatgcacgcaagccatgcctttgatattagcctgttcgaaatgtgggtgcctctggttagcggtgctcgcgtggttttggctggtagcggcgccgttgatggtgcggcactggcggcatacgtcgcagacggcgtgaccgcagcccatctgacggcaggcgcgttccgtgtcctggcagaagagagccctgagagcgtcgcgggtttgcgtgaggtgttgactggcggtgacgccgtgccgctggccgcagttgagcgcgttcgtcgtacctgcccggatgttcgtgtgcgtcacctgtatggtccgaccgaggcgacgctgtgtgcaacgtggttgctgctggaaccgggtgatgaaacgggtcctgttctgccaatcggtcgtccgctggcgggccgtcgtgtttatgtactggatggtttcctgcgtccggtgcctccgggcgttgcaggcgagctgtacgttgcgggtgcgggtgttgcacaaggttatctggagcgccctgcactgacggcggagcgttttgttgcagatccgtttgttgcgcacggtcgtatgtaccgcacgggtgacctggcatactggacgggtaagggtgcactggcatttgcaggtcgcgcagatgaccaggtgaagatccgtggttaccgtgtcgagccgggtgaaattgaagttgtcctggcgggtctgccgggtgtcggtcaagcggttgtgttggcgcgtgacgagcatctgatcggctacgcagttgcggaggccggtcatgaactggacccggtgcgcctgcgcgaacagctggcggacaccctgccggagttcatggttccggctgccgtcctggtcctgggtgagctgccgctgacggtgaacggtaaagtggatcgtcaggcattgccgggtccggacttcgcaagcaaagcggcaggccgtgctccggcaaccgacgcagaacgtgtgctgtgtggtgtttttgccgaggtgctgggcttggatcgcgtttcggtcgaagatagctttttcgaattgggtggcgatagcatcagcagcatgcaagttgccgcacgtgctcgtcgtgagggtatttctttgaccccgcgtcaggtgttcgagtatcgtaccccggaacgtctggcagcgctggctcaagaagcccaaccgacccgtcgtgcggaggtaagcggtgtgggtgagattccgctgacccctgttatgcgtgctctgggcgatgacgctgtgcgcccgaattttgcccaagcacgtgtcgtcggtacgccggcaggcctgaaccaagatagcctggtgaaagcgctgcaagctgtgctggatgttcacgacctgctgcgcgctcgcgtccagagcgacggtcgcttgattgtcgcagagccaggtgccgtgaatgcagcaggcttggtgactcgtgtggcagccgagagcggtaacctggatgagattgcggaaggtcaagtttctgcggcgatgggcaccctgaacccgagcgcaggtatcatggctcgtgttgtttggatcgatgcgggctccgatgaaccaggccgtctggcttttgtggcccaccacctggcagtggatgccgttagctggggcatcttgctgccggatctgcgtagcgcgtatgacgcggtgatcgcaggtgaaaccccagcattggaaccggcagttacgagctaccgtcagtgggcgctgcgtctggcggagcaagcccgtagcgactccacggtggctgaggttgaccaatgggttgaactgttggacggcgcagaaagcgttctggaacagcaaacgggtcagagccacagctggagcgatgcgctgtccggccctgttgcccgtaccctggtgtcccagttgccggctgcgttccactgcggcattcaggatgttctgctggcaggtttggccggtgcggtggcgcgtgtgcgcggtgccggtgctggtttgctggttgatgttgagggtcacggtcgtgatgccgccgacggtgaggacctgttgcgcaccgttggttggttcaccagcgtgcacccggtccgcttggatttggcggatctgagcttgaaagctgtcaaagaacaggtccgtgcggttcctggcgatggcttgggttatggtctgctgcgctatctgaatccggaaaccgctgcgcgtctggccggtctgccgagcgctcagattggtttcaactatctgggccgcacctccctgaccctgaaaaatccggcttgggaggtgagcggcgagggtccactgggcggtggcccggacaccgccctggcccacctggttgaagtcggtgctgaagtccaagataccccggatggtccgcgtctgggtctggccattgatggccgcgacattgatccggcgacggtccagcagctgggtgaagcgtggctggagatcctgaccgccttggcggatgacgccggtgcaggtggccacacagagaccggttctcgtagccaccatcatcaccatcactaacctgcagg

SEQ ID NO: 26 - protein, 538 aa, Amycolatopsis balhimycina
LTVAGVEVTTAAERALVAGEWGASTSAPPSLPALDLFGHQVAHRRDEPAVVDGDRTVSYGELAERAERLAGYLNGRGVRRGDRVAVVLDRSPDLIATLLAVWKAGAAYVPVDPAYPVERRKFMLADSGPAAVVCAEAYRAAVPDTCPEPIVLDDPRTRQAVAESPRLSAGTSADDLAYVMYTSGSTGTPKGVAVSHGNVAALAGEPGWRVGPGDAVLLHASHAFDISLFEMWVPLLSGARVVLAGPGAVDGAALAAYVAGGVTAAHLTAGAFRVLADESPEAVAGLREVLTGGDAVPLAAVERVRGRVRNVRVRHLYGPTEATLCATWWLLEPGDETGSVLPIGRPLAGRRVHVLDAFLRPVPPGVAGELYVAGAGVAQGYSSRPALTAERFVADPSGSGARMYRTGDLAYWTEQGALAFAGRADDQVKIRGYRVEPGEIEVVLAGLPGVGQAVVTPRGEHLIGYVVAEAGHDADPVRLREQLAGTLPEFMVPAAVLVLDELPLTVNGKVDRRALPEPDFAAKSAGREPVTEAERVLC

SEQ ID NO: 27 - protein, 538 aa, uncultured soil bacterium
LRVADVDVTSAAERELVVNEWSAASHAAPSRLAPDLFGRQVERRRDEVAVVDGDRAMSYGELAERAEKLAGYLSGRGVRRGDRVAVVMDRSPDLIATLLAVWKAGAAYVPVDPAYPVERVKFMLADAEPAAVVCAEAYRDAALDGGLDPIVLDDPRTRQAVAECTRLSVGATADDLAYVMYTSGSTGTPKGVAVSHGNVAALVGEPGWAGSPDDAVLMHASHAFDISLFEMWVPLLSGARVVLAGSGAVDGEALAGYVAGGVTAAHLTAGTFRVVAEESPESIAGLREVLTGGDAVPPAAVERVRRTCPGVRVRHLYGPTEATLCATWWLLEPGDETGSVLPIGRPLSGRRVYVLDAFLRPVPPGVAGELYVAGAGVAQGYLGRSALTAERFVADPFVPAERMYRTGDLAYWMDQGALAFAGRADDQVKIRGYRVEPGEIEVVLAGLPGVGQAVVSARDEHLIGYVVAEAGQDVDPVRLRGQLAETLPEFMVPAAVLVLDELPLTVNGKVDRQALPEPDFASKAVGREPATEAERILC

SEQ ID NO: 28 - DNA, 1661 bp, Artificial sequence
  source 1..1661: /organism=“Artificial sequence” /note=“Synthetic DNA” /mol_type=“unassigned DNA”
catatgctcaccgtcgcaggcgtcgaagttactaccgccgcagagagagcattggtggcgggtgagtggggtgcgagcacgagcgcaccgccgtccctgccggcattggatttgttcggtcatcaagtggcgcaccgtcgtgacgaaccggcggttgtggacggtgatcgtaccgttagctacggtgagctggccgaacgcgcggagcgtctggccggctacctgaacggccgtggcgttcgtcgtggtgaccgtgttgctgttgtgctggaccgtagcccggacctgattgcaaccctgctggctgtttggaaggcaggtgcggcctatgtcccggttgacccggcttaccctgtggaacgtcgtaagtttatgctggctgactctggccctgccgcggtggtgtgcgctgaggcataccgcgcagcggtgccggatacgtgtccggaaccgatcgtgctggatgatccgcgcacccgccaggctgtggcggagagcccgcgtttgagcgcaggcacctcggccgatgacctggcgtacgtgatgtacaccagcggtagcaccggcacgccgaaaggtgtagcagtgtctcatggcaacgtcgcggctctggcaggtgagcctggctggcgcgttggccctggcgacgcggtcctgctgcatgcgagccacgcctttgatattagcctgttcgagatgtgggtcccgctgctgagcggcgcacgtgttgtcctggcgggcccgggtgcagtcgatggtgcggcgctggcggcgtatgtcgcgggtggtgtgaccgccgcacacctgaccgcgggtgctttccgtgtgctggcggacgagtcgccagaggcagtagcgggcctgcgtgaagtcctgaccggcggtgatgcggtgccgctggcagcggttgaacgtgtgcgtggccgtgtccgcaatgtgcgtgttcgtcacctgtatggcccgacggaagctacgctgtgcgcgacgtggtggttgctggaaccgggtgatgagactggcagcgtcctgccgatcggtcgtccgctggcgggtcgtcgtgtccatgttctggatgcattcctgcgtccggtcccaccaggtgtcgccggtgaactgtatgttgcgggtgcaggcgttgcgcaaggttacagcagccgtccggcgctgactgccgagcgtttcgttgctgacccgtctggtagcggtgcccgcatgtatcgcacgggtgacctggcatactggaccgagcagggtgcgctggcctttgcaggtcgtgctgacgatcaagtcaaaattcgcggttatcgcgttgaaccgggcgaaattgaagtggtgctggcaggtttgccgggtgtgggtcaagcggtcgtgacgccgcgtggtgaacatctgatcggttacgttgtggccgaagcgggtcacgatgcggaccctgttcgcctgcgcgaacagctggcgggcaccctgccggagtttatggtcccggcagccgtgctggtgttggatgagctgccgctgaccgttaatggtaaagttgaccgtcgcgcgctgccggagccggatttcgcggccaagtccgccggtcgcgagccggtcacggaggcggagcgcgttctgtgtggcagccgcagccaccaccatcatcaccactaacctgcagg

SEQ ID NO: 29 - DNA, 1661 bp, Artificial sequence
  source 1..1661: /organism=“Artificial sequence” /note=“Synthetic DNA” /mol_type=“unassigned DNA”
catatgctgagagttgccgacgtcgacgtcacgagcgctgccgagagagagctggtcgtcaacgaatggagcgcagcgagccatgcagccccgtcccgtctggcaccagacctgtttggccgtcaagttgaacgccgtcgtgacgaagttgccgttgttgatggcgatcgtgcgatgagctatggcgagctggccgaacgcgctgaaaaactggccggctatctgagcggtcgcggtgttcgccgtggtgaccgtgtggcggtggttatggaccgcagcccggacctgatcgctacgctgctggcggtgtggaaggctggtgcggcatacgtcccggttgacccggcatacccggttgagcgcgttaagttcatgctggcggatgcggagccagctgcggtggtctgcgcggaagcgtatcgcgacgcggcgttggatggtggtctggacccgattgttttggatgatccgcgtacccgccaagcagttgcggagtgcacccgtctgagcgtgggtgcgactgcggatgacctggcttacgtgatgtataccagcggcagcactggcacgccgaagggtgtcgccgttagccacggcaatgtcgccgcgttggtgggtgagccgggctgggcgggttccccggacgacgcagttttgatgcacgcatcccatgcattcgacatcagcctgtttgagatgtgggttccgctgttgagcggtgcacgtgttgttctggcgggtagcggtgccgtcgatggcgaggcactggcaggttacgtagccggtggtgtcacggccgcacacctgacggcaggcacctttcgtgtggtagcggaagagtctccagaaagcatcgccggtctgcgtgaggtgctgacgggtggcgacgcggtcccgccagcggcggtggagcgcgtccgtcgcacctgtccgggcgttcgcgtgcgtcacctgtacggtcctaccgaggcgacgctgtgcgcgacctggtggttgctggagccgggtgacgaaaccggctccgtgctgccgattggccgtccgctgagcggccgtcgcgtctacgttctggacgcctttctgcgtccggtgccaccgggtgttgccggtgaactgtacgtggccggtgccggcgtagcgcagggctatctgggccgcagcgcgttgaccgcagaacgttttgtcgcggacccgttcgtgcctgctgaacgtatgtatcgtaccggcgatctggcgtattggatggatcagggtgcactggcgttcgcaggtcgtgctgatgatcaggtgaaaattcgcggttaccgcgtggaaccgggtgagattgaggtcgtcctggcgggtttgccgggtgtgggccaggcggttgtgagcgcccgtgacgagcatttgatcggttacgtcgtggcggaagctggtcaggatgttgacccagtccgtctgcgtggtcaactggcggagactctgccggagttcatggttccggcagcggtgctggtcctggatgaactgccgctgaccgtgaacggtaaagtggatcgtcaagcactgccggagccggatttcgcatccaaagcggtcggccgtgagccggcgaccgaagcagagcgtatcctgtgtggcagccgttcgcatcatcaccaccaccactaacctgcagg

SEQ ID NO: 30 - protein, 73 aa, Streptomyces lavendulae
MTNPFDNENGTFLVLVNDEGQHSLWPVFAEIPQGWTTAFGEASRAECLEFVEQNWTDMRPKSLVARMEGTATA

SEQ ID NO: 31 - protein, 69 aa, Amycolatopsis balhimycina
MSNPFDNEDGSFFVLVNDEGQHSLWPTFAEVPAGWTRVHGEAGRQECLAYVEENWTDLRPKSLIREASA

SEQ ID NO: 32 - protein, 69 aa, uncultured soil bacterium
MTNPFDNEDGSFFVLVNDEGQHSLWPTFAEVPAGWVCVYGEATRQECLTFVEENWTDLRPKSLIQEVGG

SEQ ID NO: 33 - DNA, 275 bp, Artificial Sequence
  source 1..275: /organism=“Artificial Sequence” /note=“Synthetic DNA” /mol_type=“unassigned DNA”
ggatccaggaggacagctatgaccaacccatttgacaacgaaaacggaacattcttagtattagtaaacgacgaaggtcagcacagcctgtggccggtctttgcagagatcccgcaaggttggacgaccgcgttcggcgaggcgtcccgcgctgagtgcctggagttcgttgagcagaattggaccgatatgcgtccgaaaagcctggtggcgcgtatggaaggtaccgccacggcaccgggcggccatcatcatcatcatcattgacctgcagg

SEQ ID NO: 34 - DNA, 263 bp, Artificial sequence
  source 1..263: /organism=“Artificial sequence” /note=“Synthetic DNA” /mol_type=“unassigned DNA”
ggatccaggaggacagctatgagtaacccatttgataatgaggacggtagtttctttgtgttagtgaatgatgaaggtcagcacagcctgtggccgaccttcgctgaggttccggcaggttggacgcgtgtccatggcgaggcaggccgtcaagagtgcctggcgtacgttgaagagaactggaccgacctgcgcccgaaaagcctgatccgtgaagccagcgcgccgggcggccatcatcatcatcatcattgacctgcagg

SEQ ID NO: 35 - DNA, 263 bp, Artificial Sequence
  source 1..263: /organism=“Artificial Sequence” /note=“Synthetic DNA” /mol_type=“unassigned DNA”
ggatccaggaggacagctatgacgaacccatttgataatgaggacggtagtttctttgtacttgtgaacgatgaaggtcagcacagcctgtggccgaccttcgcagaggttccggctggctgggtgtgcgtctacggtgaagcgacccgtcaggagtgtctgacgttcgttgaagagaattggaccgacctgcgcccgaaaagcctgatccaagaggtcggcggtccgggcggccatcatcatcatcatcattgacctgcagg

(19) United States
(12) Patent Application Publication
(10) Pub. No.: US 2014/0349340 A1
(43) Pub. Date: Nov. 27, 2014

(54) MBTH-LIKE PROTEINS IN THE PRODUCTION OF SEMI SYNTHETIC ANTIBIOTICS

(71) Applicant: DSM SINOCHEM PHARMACEUTICALS NETHERLANDS B.V., Delft (NL)

(72) Inventors: Ulrike Maria Mueller, Echt (NL); Rémon Boer, Echt (NL); Roelof Ary Lans Bovenberg, Echt (NL)

(21) Appl. No.: 14/374,137

(22) PCT Filed: Jan. 28, 2013
(86) PCT No.: PCT/EP2013/051544

§ 371 Date: Jul. 23, 2014

(30) Foreign Application Priority Data
Jan. 31, 2012 (EP) 12153225.3

(51) Int. Cl. C07K 5/083

(52) U.S. Cl. CPC C07K 5/0806
USPC 435/68.1; 435/254.5; 435/254.11; 435/254.3

(57) ABSTRACT

The present invention relates to the preparation of β-lactam antibiotics comprising contacting 4-hydroxyphenylglycine or phenylglycine, cysteine and valine with a non-ribosomal peptide synthetase and subsequent cyclization using an isopenicillin N synthase in the presence of an MbtH-like protein and to a host cell equipped to perform such preparation.

### FIELD OF THE INVENTION

The present invention relates to the preparation of β-lactam antibiotics comprising contacting 4-hydroxyphenylglycine or phenylglycine, cysteine and valine with a non-ribosomal peptide synthetase and subsequent cyclization using an isopenicillin N synthase in the presence of an MbtH-like protein and to a host cell equipped to perform such preparation.

### BACKGROUND OF THE INVENTION

MbtH-like proteins are small proteins resembling MbtH from Mycobacterium tuberculosis. The function of MbtH-like proteins is, to a large extent, still unknown although recent studies indicate a role in the biosynthesis of peptides, in particular in the stimulation of adenylation reactions. Heemstra et al. (J. Amer. Chem. Soc. (2009) 131, 15317-15329) have reported adenylation of N(5)-((R)-3-hydroxybutyryl)-N(5)-hydroxy-D-ornithine using the adenylation domain VbsS whereby involvement of the MbtH-like protein VbsG was shown. Likewise, Felnagle et al. (Biochemistry (2010) 49, 8815-8817) have reported the adenylation of L-serine, β-lysine and L-2,3-aminopropionic acid using the adenylation domains EntF, CmnO/VioO and CmnA respectively. For L-serine adenylation the MbtH-like protein YbdZ was shown to be involved, for β-lysine these were CmnN or VioN whereas CmnN was also found to be involved in adenylation of L-2,3-aminopropionic acid. In addition MbtH-like proteins KtzJ, PacJ and GIbE were shown by Zhang et al. (Biochemistry (2010) 49, 9946-9947) to be involved in the adenylation of m-tyrosine using the adenylation domain PacL and finally it was demonstrated by Boll et al. (J. Biol. Chem. (2011) 286, 36281-36290) that MbtH-like proteins CloY, SimY and Orf1van are involved in adenylation of L-tyrosine by adenylation domains CloH, SimH or Pcza361.18.

The genes encoding MbtH-like proteins, mbtH-like genes, are often found in non-ribosomal peptide synthetase (NRPS) gene clusters of prokaryotic microorganisms. Many mbtH-like genes are deposited in Gen Bank. In order to identify MbtH-like proteins a BLASTP study shows homologues encoded by members of Actinobacteria, Firmacutes and Proteobacteria, however not by Archaea (R. H. Baltz, J. Ind. Microbiol. Biotechnol. (2011) 38, 1747-1760). There are no reports of mbtH-like genes in eukaryotic organisms.

Of the secondary metabolites produced by microorganisms, many are of significant value. An important class in this respect is that of the β-lactam antibiotics, notably the penicillins and cephalosporins. The first step in the biosynthesis of the penicillin antibiotics is the condensation of the L-isomers of three amino acids, L-α-amino adipic acid (A), L-cysteine (C) and L-valine (V) into a tripeptide, δ-(L-α-aminoadipyl)-L-cysteinyl-D-valine (ACV). This step is catalyzed by δ-(L-α-aminoadipyl)-L-cysteinyl-D-valine synthetase (ACVS). In the second step, ACV is oxidatively cyclized by the action of isopenicillin N synthase (IPNS). The product of this reaction is isopenicillin N from which the penicillins G or V are formed by exchange of the hydrophilic α-aminoadipyl side chain by a hydrophobic side chain. The side chains commonly used in industrial processes are phenylacetic acid, yielding penicillin G, or phenoxyacetic acid, yielding penicillin V. The exchange reaction is catalyzed by the enzyme acyltransferase. Due to the substrate specificity of the enzyme acyltransferase, it is hardly possible to exchange the a-aminoadipyl side chain for any other side chain of interest, although it was shown that adipic acid and certain thio-derivatives of adipic acid could be exchanged (WO 95/04148 and WO 95/04149). In particular, the side chains of industrially important penicillins and cephalosporins cannot be directly exchanged via acyltransferase. Consequently, most of the β-lactam antibiotics presently used are prepared by semi synthetic methods. These semi synthetic β-lactam antibiotics are obtained by modifying an N-substituted β-lactam product by one or more chemical and/or enzymatic reactions. These semi synthetic methods have the disadvantage that they include many steps, are not environmentally friendly and are costly. It would therefore be highly desirable to avail of a completely fermentative route to β-lactam antibiotics, for instance to amoxicillin, ampicillin, epicillin, cefadroxil, cephalexin and cephradine.

Various options can be thought of for such a completely fermentative route to semi synthetic penicillins and cephalosporins. In WO 2008/040731 it is suggested to modify the first two steps in the penicillin biosynthetic route such that amoxicillin is directly synthesized and secreted. For instance, for amoxicillin, a tripeptide comprising the amoxicillin side chain, i.e. D-4-hydroxyphenylglycyl-L-cysteinyl-D-valine, is constructed instead of ACV which is subsequently cyclized with a modified IPNS.

ACVS is an NRPS that catalyses the formation of the tripeptide LLD-ACV. In this tripeptide, a peptide bond is formed between the δ-carboxylic group of L-α-aminoadipic acid and the amino group of L-cysteine, and additionally the conformation of valine is changed from L to D. WO 2008/040731 discloses a modified ACVS capable of catalyzing the formation of L-4-hydroxyphenylglycyl-L-cysteinyl-D-valine and L-phenylglycyl-L-cysteinyl-D-valine (precursor for ampicillin) and capable of modifying the L stereochemical configuration of the first amino acid into a D configuration.

WO 2008/040731 also discloses that native and engineered IPNS is capable of acting on D-4-hydroxyphenylglycyl-L-cysteinyl-D-valine and D-phenylglycyl-L-cysteinyl-D-valine.

Preferably the above approach is carried out in an organism capable of production under industrial conditions such as eukaryotes like Aspergillus and Penicillium. A problem associated with this approach is that yields are still low and require significant improvement.

### DETAILED DESCRIPTION OF THE INVENTION

In the context of the present invention, the term “adenylation domain” refers to a protein sequence capable of recognition and activation of a specific amino acid. Preferred adenylation domains are derived from non-ribosomal peptide synthetases capable of incorporating the respective amino acids. The term “N-α-amino-4-hydroxyphenylacetyl β-lactam antibiotic” refers to β-lactam antibiotics having a 4-hydroxyphenylglycine side chain such as amoxicillin, cefadroxil, cefatrizine, cefoperazone, cefpiramide, cefprozil, intermediates thereto and the like, preferably amoxicillin.

The term “N-α-aminophenylacetyl β-lactam antibiotic” refers to β-lactam antibiotics having a phenylglycine side chain such as ampicillin, cefaclor, cephalexin, cephaloglycine, intermediates thereto and the like, preferably ampicillin.

The term “module” defines a catalytic unit that enables incorporation of one peptide building block, usually an amino acid, in the product, usually a peptide, and may include domains for modifications like epimerization and methylation.

The term “heterologous” used in combination with modules refers to modules wherein domains, such as adenylation or condensation domains, are from different modules. These different modules may be from the same enzyme or may be from different enzymes.

The term “specific for” indicates that a module referred to as being specific for enables incorporation of the indicated amino acid.

In a first aspect of the invention there is disclosed a method for the preparation of an N-α-amino-4-hydroxyphenylacetyl or an N-α-aminophenylacetyl β-lactam antibiotic comprising the steps of:
(a) contacting the amino acids 4-hydroxyphenylglycine or phenylglycine, cysteine and valine with a non-ribosomal peptide synthetase (NRPS) to give a tripeptide 4-hydroxyphenylglycyl-cysteinyl-valine or a tripeptide phenylglycyl-cysteinyl-valine, respectively;
(b) contacting the tripeptide obtained in step (a) with an isopenicillin N synthase,
   whereby an MbtH-like protein is present.

Addition of MbtH-like proteins to improve adenylation in vitro and in vivo in their original prokaryotic hosts has been implied in R. H. Baltz (J. Ind. Microbiol. Biotechnol. (2011) 38, 1747-1760), Felnagle et al. (Biochemistry (2010) 49, 8815-8817), Wenjum Zhang et al. (Biochemistry (2010) 49, 9946-9947) and Boll et al. (J. Biol. Chem. (2011) 286, 36281-36290), however these documents do not indicate that such an approach may be successful in eukaryotes nor is there an indication of the use of MbtH-like proteins in β-lactam antibiotics. In general, involvement of MbtH-like proteins in incorporation of hydroxyphenylglycine or phenylglycine has hitherto not been reported. In contrast, Stegman et al. (FEMS Microbial Letter (2006) 262, 85-92) discloses the opposite, namely that the small MbtH-like protein encoded by an internal gene of the balhimycin biosynthetic gene cluster is not required for glycopeptide production by Amycolatopsis balhimycina, a glycopeptide comprising hydroxyphenylglycine. Hence, the prior art does not provide any pointers towards the use of MbtH-like proteins in the preparation of an N-α-amino-4-hydroxyphenylacetyl or an N-α-aminophenylacetyl β-lactam antibiotic. Surprisingly it was found that the incorporation of L-hydroxyphenylglycine or L-phenylglycine by the adenylation domains of the present invention is possible only in the presence of an MbtH-like protein.

In a first embodiment, preferred MbtH-like proteins are the ones described in R. H. Baltz (J. Ind. Microbiol. Biotechnol. (2011) 38, 1747-1760). More preferred MbtH-like proteins are the ones comprising invariant amino acids N17, E19, Q21, S23, W25, P26, P32, G34, W35, L48, W55, T56, D57, R59 and P60, also suitably referred to with the amino acid code NXEXQXSXWP-X₅-PXGW-X₁₃-L-X₇-WTDXRP. In the io above annotation the letters D, E, G, L, N, P, Q, R, S, T, W and X refer to the commonly known single letter codes for amino acids (whereby X denotes one unspecified amino acid, X₅ denotes 5 unspecified amino acids, X₇ denotes 7 unspecified amino acids and X₁₃ denotes 13 unspecified amino acids). Preferably, the MbtH-like proteins of the present invention are those that are present in the biosynthesis clusters of which module M1 (see below) is chosen. Most preferred are Tcp13 (SEQ ID NO: 18) or Tcp17 (SEQ ID NO: 19) obtained from the teicoplanin biosynthesis cluster from Actinoplanes teichomyceticus (Sosio et. al., Microbiology (2004) 150, 95-102), or the MbtH-like homologue identified in the Veg biosynthesis cluster obtainable from an uncultured soil bacterium (Banik J. J. and Brady S. F., Proc. Natl. Acad. Sci. USA (2008) 105, 17273-17277) encoded by nt 33826-34035 of GenBank: EU874252 (SEQ ID NO: 20) or the MbtH-like homologue identified in the Teg biosynthesis cluster obtainable from an uncultured soil bacterium (Banik J. J. and Brady S. F., Proc. Natl. Acad. Sci. USA (2008) 105, 17273-17277) encoded by nt 33949-33158 of GenBank: EU874253 (SEQ ID NO: 32) or the MbtH-like homologue (SEQ ID NO: 31) identified in the balhimycin biosynthesis cluster from Actinoplanes balhimycina (Recktenwald et al., Microbiology (2002) 148, 1105-1118, Stegman et al., FEMS Microbial Lett. (2006) 262, 85-92) or the MbtH-like homologue (SEQ ID NO: 30) identified in the complestatine biosynthesis cluster from Streptomyces lavendulae (Chiu et al., Proc. Natl. Acad. Sci. USA (2001) 98, 8548-8553) or MbtH-like proteins having an amino sequence with a percentage identity of at least 30%, more preferably at least 40%, even more preferably at least 50%, most preferably at least 60% to said sequences. Such polypeptide modules with a percentage identity of at least 30% are also called homologous sequences or homologues.

The adenylation domain of a module determines specificity for a particular amino acid as it is responsible for recognition and activation of a dedicated amino acid and its loading of the correct amino acid onto its downstream adjacent partner thiolation domain. The adenylation reaction catalyzed by the adenylation domain is the following:

Amino acid+ATP [symbol] aminoacyl-AMP+PPi.

ATP, Mg²⁺, and amino acid are sequentially bound reversibly to the adenylation domain. Subsequently reversible breakdown of ATP by the adenylation domain into AMP is mediated by the amino acid. In this last step PPi is released. Several suitable methods for the determination of adenylation specificity are known in the art.

The classical radioactive ATP-[³²P] pyrophosphate (PPi) exchange assay (Santi et al. (Meth. Enzymol. (1974) 29, 620-627) is a common method for adenylation domain specificity determination. This method exploits the reverse reaction of AMP to ATP to quantify the interaction between the adenylation domain and the respective substrate. It uses the formation of isotopically labeled ATP, which is formed when [³²P]PPi is incorporated into AMP. The increase in labeled ATP is measured to detect the adenylation reaction (for example Recktenwald et al. (2002) Microbiology 148, 1105-1118). For the purpose of the present invention, pyrophosphate formation is analyzed using a more recently developed assay that measures the release of PPi with a method that does not require radioactive phosphates. These assays use inorganic pyrophosphatases to convert PPi produced during aminoacyl-AMP formation to orthophosphate (Pi). To measure Pi concentrations some of these assays use molybdate/malachite green reagent for colorimetric detection (McQuade et al. 2008) or, as used in the context of the present invention, a shift in absorbance maximum by conversion of 7-methyl-6-thioguanosine (MESG) by purine nucleoside phosphorylase (Ehmann D. E. et al. (Proc. Natl. Acad. Sci. (2000) 97, 2509-2514) or Daniel & Aldrich (Anal. Biochem. (2010) 404, 56-63)).

In order to perform these assays the corresponding enzymes preferably are present as purified proteins. Several methods are available to the skilled person in order to obtain these purified proteins. These include the heterologous over expression of the whole module comprising the adenylation domain or its single adenylation domain in a suitable host organisms like Escherichia coli or Streptomyces lividans as for example disclosed by Recktenwald et al. (Microbiology (2002) 148, 1105-1118). Preferably, these domains or modules are equipped with a tag to be used for purification by affinity chromatography. As known to the skilled person in the art these tags are useful for the characterization of the enzymes but not needed for their performance in the suitable host.

In a second embodiment, the NRPS constructs of the present invention comprise three modules, a first module M1 specific for 4-hydroxyphenylglycine and/or phenylglycine, a second module M2 specific for cysteine and a third module M3 specific for valine. The first module M1 enables incorporation of a first amino acid L-4-hydroxyphenylglycine or L-phenylglycine and, preferably, its conversion to the corresponding D-amino acid. The second module M2 enables incorporation of the amino acid L-cysteine while being coupled to the amino acid 4-hydroxyphenylglycine or phenylglycine. In particular, when the amino acid 4-hydroxyphenylglycine or phenylglycine is in its D-form, the M2 module specific for cysteine comprises a condensation domain that is D-specific for the donor and L-specific for the acceptor (^{D}C_{L}) that is fused to an adenylation domain that is heterologous thereto. The third module M3 enables incorporation of the amino acid L-valine and its conversion to the corresponding D-amino acid. In this way, the NRPS catalyzes the formation of a DLD-tripeptide D-4-hydroxyphenylglycyl-L-cysteinyl-D-valine or D-phenylglycyl-L-cysteinyl-D-valine from their L-amino acid precursors.

Each NRPS module is composed of so-called “domains”, each domain being responsible for a specific reaction step in the incorporation of one peptide building block. Each module at least contains an adenylation domain, responsible for recognition and activation of an amino acid and a thiolation domain, responsible for transport of intermediates to the catalytic centers. The second and further modules in addition contain a condensation domain, responsible for formation of the peptide bond and the last module further contains a termination domain, responsible for release of the peptide. Optionally, a module may contain domains such as an epimerization domain, responsible for conversion of the L-form of the incorporated amino acid to the D-form. See Sieber et al. (Chem. Rev. (2005) 105, 715-738) for a review of the modular structure of NRPS.

In a third embodiment, a suitable source for the M1 module of the hybrid peptide synthetase of the present disclosure is an NRPS catalyzing formation of a peptide comprising the amino acid 4-hydroxyphenylglycine or phenylglycine to be incorporated as first amino acid in the peptide. Thus, a suitable M1 module is selected taking into account the nature of the amino acid to be incorporated as first amino acid of the tripeptide. In particular, the adenylation domain of a module determines selectivity for a particular amino acid. Thus, an M1 module may be selected based on the specificity of an adenylation domain for the amino acid to be incorporated. Such a selection may occur according to the specificity determining signature motif of adenylation domains as defined by Stachelhaus et al. (Chem. & Biol. (1999) 6, 493-505) and by Rausch et al. (Nucleic Acids Res. (2005), 33, 5799-5808). The M1 module does not need to contain a condensation domain or a termination domain as it is the first module of the NRPS. Thus, if present in the source module, condensation and/or termination domains may suitably be removed to obtain a first module M1 without said domains. In addition to an adenylation and a thiolation domain, the module M1 NRPS should contain an epimerization domain if an L-amino acid needs to be converted to a D-amino acid. Thus, if not present in the source module, an epimerization domain is fused to the thiolation domain of the source module to obtain a first module M1 containing adenylation, epimerization and termination domains.

Preferably, a first module M1 with 4-hydroxyphenylglycine specificity is obtainable from 4-hydroxyphenylglycine specific modules from synthetases involved in the formation of the glycopeptide antibiotic vancomycin or of the vancomycin-class compounds chloroeremomycin or balhimycin, a vancomycin synthetase, chloroeremomycin synthetase or balhimycin synthetase. Preferred modules are the fourth and fifth module of a vancomycin synthetase, chloroeremomycin synthetase, balhimycin synthetase or Veg synthetase, (and the first and the third module Veg synthetase). Preferred sources are chloroeremomycin synthetase obtainable from Amycolatopsis orientalis (Trauger et al., Proc. Nat. Acad. Sci. USA (2000) 97, 3112-3117), balhimycin synthetase obtainable from Amycolatopsis balhimycina (formerly Amycolatopsis mediterranei) Blp-Cluster (Recktenwald et al., Microbiology (2002) 148, 1105-1118) and Veg synthetase obtainable from an uncultured soil bacterium Veg-cluster (Banik J. J. and Brady S. F., Proc. Natl. Acad. Sci. USA (2008) 105, 17273-17277). Alternatively, 4-hydroxyphenylglycine specific modules may be obtained from synthetases involved in the formation of the lipoglycopeptide antibiotic teicoplanin or teicoplanin-class antibiotics as A47934, A40926 or Teg, a teicoplanin synthetase, A47934 synthetase, A40926 synthetase or Teg synthetase. Preferred modules are the first, fourth and fifth module of a teicoplanin synthetase, A47934 synthetase, A40926 synthetase or Teg synthetase. Preferably these modules are obtained from teicoplanin synthetase from Actinoplanes teichomyceticus Tcp-cluster (Sosio et. al., Microbiology (2004) 150, 95-102), A47934 synthetase obtainable from Streptomyces toyocaensis NRRL15009 Sta-Cluster, A40926 synthetase obtainable from Nanomurea sp. ATCC39727 Dbv-Cluster (Sosio et. al., Chem. Biol. (2003) 10, 541-549) or a Teg synthetase obtainable from an uncultured soil bacterium Teg-cluster (Banik J. J. and Brady S. F., Proc. Natl. Acad. Sci. USA (2008) 105, 17273-17277). Alternatively, 4-hydroxyphenylglycine specific modules may be obtained from a complestatin synthetase, in particular the seventh module of a complestatin synthetase, preferably a complestatin synthetase obtainable from Streptomyces lavendulae (Chiu et al., Proc. Nat. Acad. Sci. USA (2001) 98, 8548-8553); Alternatively, a first module M1 with 4-hydroxyphenylglycine specificity is obtained from a CDA (Calcium-Dependent Antibiotic) synthetase and is in particular the sixth module of a CDA synthetase whereby the numbering of CDA synthetase modules as published by Hojati et al. (Chem. & Biol. (2002) 9, 1175-1187) is used. Preferably, the CDA synthetase is obtained from Streptomyces coelicolor.

Alternatively, for the preparation of an N-α-aminophenylacetyl β-lactam antibiotic, a first module M1 with phenylglycine specificity may be obtained from a pristinamycin synthetase, in particular the C-terminal module of the SnbD protein of pristinamycin synthetase, as published by Thibaut et al. (J. Bact. (1997) 179, 697-704). Preferably, the pristinamycin synthetase is obtainable from Streptomyces pristinaspiralis. The C-terminal source module from pristinamycin synthetase contains a termination domain and does not contain an epimerization domain. To prepare a module functioning as a first module in the peptide synthetase of the invention, the termination domain suitably is removed from the C-terminal source module and an epimerization domain is fused to the thiolation domain of the thus-modified C-terminal module. An epimerization domain may be obtainable from any suitable NRPS, for instance from another module of the same NRPS enzyme or from a module of a different NRPS enzyme with similar (e.g. 4-hydroxyphenylglycine or phenylglycine) or different amino acid specificity of the adenylation domain. Preferably, the epimerization domain is obtainable from a CDA Synthetase from Streptomyces coelicolor, more preferably from the sixth module, as specified above. Thus, in this embodiment, the module M1 of the NRPS is a hybrid module. The epimerization domains described above may also be fused to those modules M1 with 4-hydroxyphenylglycine specificity lacking an epimerization domain as described in the first embodiment.

Unexpectedly, it is found that several modules M1 with 4-hydroxyphenylglycine specificity as described in the first embodiment are capable of activating L-phenylglycine in the presence of MbtH-like proteins and are therefore suitable for use as first module M1 in the construction of NRPS constructs designed for N-α-aminophenylacetyl β-lactam antibiotics. These modules are for example the first module of a teicoplanin synthetase, A47934 synthetase or A40926 synthetase. Preferably these first modules are obtained from teicoplanin synthetase from Actinoplanes teichomyceticus Tcp-cluster (Sosio et. al., Microbiology (2004) 150, 95-102), A47934 synthetase obtainable from Streptomyces toyocaensis NRRL15009 Sta-Cluster or A40926 synthetase obtainable from Nanomurea sp. ATCC39727 Dbv-Cluster (Sosio et. al., Chem. Biol. (2003) 10, 541-549). These modules are further the third module of a teicoplanin synthetase, or a Veg synthetase. Preferably, these first modules are obtained from teicoplanin synthetase from Actinoplanes teichomyceticus Tcp-cluster (Sosio et. al., Microbiology (2004) 150, 95-102), or Veg synthetase obtainable from an uncultured soil bacterium Veg-cluster (Banik J. J. and Brady S. F., Proc. Natl. Acad. Sci. USA (2008) 105, 17273-17277). These modules are further the fifth module of a chloroeremomycin synthetase, or balhimycin synthetase. Preferred sources for the fifth module are chloroeremomycin synthetase obtainable from Amycolatopsis orientalis (Trauger et al., Proc. Nat. Acad. Sci. USA (2000) 97, 3112-3117), and balhimycin synthetase obtainable from Amycolatopsis balhimycina (formerly Amycolatopsis mediterranei) Blp-Cluster (Recktenwald et al., Microbiology (2002) 148, 1105-1118).

In a fourth embodiment, the second module M2 of the peptide synthetase should enable incorporation of the amino acid cysteine as second amino acid of the tripeptide DLD-XCV, wherein X is 4-hydroxyphenylglycine or phenylglycine. Selection of this module may be based on the specificity determining signature motif of adenylation domains as published by Stachelhaus et al. (Chem. & Biol. (1999) 8, 493-505). An example for the second module M2 is the first module of the peptide synthetase Ecm7 which naturally incorporates N-Me-L-Cys-N-Me-L-Val in echninomycin (a quinomycin antibiotic) biosynthesis by Streptomyces lasaliensis (Watanabe et al. in Nat. Chem. Biol. (2006) 2, 423-428), whereby the N-methylation activity of Ecm7 is removed by mutation as described by Watanabe et al. (Curr. Opin. Chem. Biol. (2009) 13, 189-196).

To enable coupling of the L-cysteinyl acceptor to the D-X-aminoacyl donor, the condensation domain of the M2 module is a ^{D}C_{L} domain, as outlined above and as explained in Clugston et al. (Biochemistry (2003) 42, 12095-12104). This ^{D}C_{L} domain is fused to an adenylation domain that is heterologous thereto. The hybrid M2 module comprising such a ^{D}C_{L}-adenylation domain configuration appears capable of incorporation of the amino acid cysteine. In a preferred embodiment, the ^{D}C_{L} domain of the M2 module is obtainable from the module immediately downstream of the module that is the source of the first module M1 of the peptide synthetase of the invention. For instance, the ^{D}C_{L} domain of the M2 module of the peptide synthetase is the ^{D}C_{L} domain of the seventh module of the CDA synthetase that is the source of the first module M1. In another embodiment, the ^{D}C_{L} domain of the M2 module of the peptide synthetase is the ^{D}C_{L} domain of the second module of the Bacillus subtilis RB14 Iturin Synthetase Protein ItuC, as defined by Tsuge et al. (J. Bacteriol. (2001) 183, 6265-6273). In a preferred embodiment of the invention, the second module M2 of the peptide synthetase is at least partly obtainable from the enzyme that is the source of the third module M3 of the peptide synthetase. In particular, the adenylation and thiolation domains of the M2 module of the peptide synthetase are obtainable from the module immediately upstream of the module that is the source of the third module of the peptide synthetase of the invention. For instance, the adenylation and thiolation domains of the M2 module of the peptide synthetase may be the adenylation and thiolation domains of the second module of an ACVS.

In a fifth embodiment, the third module M3 of the peptide synthetase enables incorporation of the amino acid valine as the third amino acid of the tripeptide, as well as its conversion to the D-form, to yield the tripeptide DLD-XCV. An example for the third module M3 is the second module of the peptide synthetase Ecm7 which naturally incorporates N-Me-L-Cys-N-Me-L-Val in echninomycin by Streptomyces lasaliensis (Watanabe et al. in Nat. Chem. Biol. (2006) 2, 423-428), whereby the N-methylation activity of Ecm7 is removed by mutation as described by Watanabe et al. (Curr. Opin. Chem. Biol. (2009) 13, 189-196) and an epimerization domain is fused to the thiolation domain. An epimerization domain may be obtainable from any suitable NRPS, for instance from another module of the same NRPS enzyme or from a module of a different NRPS enzyme with similar (e.g. L-valine) or different amino acid specificity of the adenylation domain. In a preferred embodiment of the invention, the third module of the peptide synthetase is obtainable from an ACVS and preferably is the third module of an ACVS. The ACVS as mentioned above preferably is a bacterial or fungal ACVS, more preferably a bacterial ACVS obtainable from Nocardia lactamdurans or a fungal ACVS obtainable from a filamentous fungus such as Penicillium chrysogenum, Acremonium chrysogenum, and Aspergillus nidulans.

The modules M1, M2 and M3 of the peptide synthetase may have the amino io acid sequences as disclosed in WO 2008/040731. Hence, the M1 module of the peptide synthetase for instance has an amino acid sequence according to SEQ ID NO: 2 or SEQ ID NO: 4 of WO 2008/040731, or contains SEQ ID NO: 1-SEQ ID NO: 9 of the present invention, or has an amino sequence with a percentage identity of at least 30%, more preferably at least 40%, even more preferably at least 50%, most preferably at least 60% to said sequences. Such polypeptide modules with a percentage identity of at least 30% are also called homologous sequences or homologues. Likewise, the M2 module of the peptide synthetase for instance has an amino acid sequence according to SEQ ID NO: 6 or to SEQ ID NO: 8 of WO 2008/040731 or an amino sequence with a percentage identity of at least 30%, more preferably at least 40%, even more preferably at least 50%, most preferably at least 60% to said sequences.

Finally, the M3 module of the peptide synthetase for instance has an amino acid sequence according to SEQ ID NO: 10 of WO 2008/040731 or an amino sequence with a percentage identity of at least 30%, more preferably at least 40%, even more preferably at least 50%, most preferably at least 60% to said sequence.

The modules of the NRPS constructs of the present invention may be obtained as disclosed in WO 2008/040731. Typically, the adenylation domain of a module determines specificity for a particular amino acid; whereas epimerization and condensation domains may be obtained form any module of choice. Engineered NRPS enzymes may be constructed by fusion of the appropriate domains and/or modules in the appropriate order. It is also possible to exchange a module or domain of an enzyme for a suitable module or domain of another enzyme. This fusion or exchange of domains and/or modules may be done using genetic engineering techniques commonly known in the art. Fusion of two different domains or modules may typically be done in the linker regions that are present in between modules or domains. See for instance EP 1255816 and Mootz et al. (Proc. Natl. Acad. Sci. USA, (2000) 97, 5848-5853) disclosing these types of constructions. Part or all of the sequences may also be obtained by custom synthesis of the appropriate polynucleotide sequence(s).

For instance, the fusion of an adenylation-thiolation-epimerization tri-domain fragment from a 4-hydroxyphenylglycine specific NRPS module to the bi-modular cysteine-valine specific fragment of an ACVS may be done by isolation using restriction enzyme digestion of the corresponding NRPS gene at the linker positions, more specifically, between the condensation domain and the adenylation domain of the 4-hydroxyphenylglycine specific module, in case of a C-terminal module or between the condensation domain and the adenylation domain of the 4-hydroxyphenylglycine specific module and between the epimerization domain and the subsequent domain (condensation or termination domain), in case of an internal elongation module. The bi-modular cysteine-valine specific fragment of ACVS may be obtained by 1) leaving the C-terminus intact, and 2) exchanging the condensation domain of the cysteine specific module 2 for a condensation domain which has ^{D}C_{L} specificity. In analogy to isolation of the adenylation-thiolation-epimerization fragment, an adenylation-thiolation-epimerization-condensation four-domain fragment may be isolated including the condensation domain of the adjacent downstream module. The latter is fused to the bi-modular cysteine-valine specific fragment of ACVS without the upstream condensation domain.

In a sixth embodiment, the NRPS enzymes as described herein may be suitably subjected to mutagenesis techniques, e.g. to improve the catalytic properties of the enzymes. Polypeptides as described herein may be produced by synthetic means although usually they will be made recombinantly by expression of a polynucleotide sequence encoding the polypeptide in a suitable host organism. Polynucleotides encoding the NRPS constructs of the present invention, polypeptides with improved activity and vectors comprising said polynucleotides are obtained as described in WO 2008/040731.

In a second aspect of the invention there is provided a host cell transformed with or comprising a polynucleotide or vector as described in WO 2008/040731 combined with a polynucleotide according to the present invention allowing the expression of an MbtH-like protein. Suitable host cells are host cells that allow for a high expression level of a polypeptide of interest. Such host cells are usable in case the polypeptides need to be produced and further to be used, e.g. in in vitro reactions. A heterologous host may be chosen wherein the polypeptides of the invention are produced in a form that is substantially free from other polypeptides with a similar activity as the polypeptide of the invention. This may be achieved by choosing a host that does not normally produce such polypeptides with similar activity. Suitable host cells also are cells capable of production of β-lactam compounds, preferably host cells possessing the capacity to produce β-lactam compounds in high levels. The host may be selected based on the choice to produce a penicillin or cephalosporin compound.

In one embodiment, a suitable host cell is a cell wherein the native genes encoding the ACVS and/or IPNS enzymes are inactivated, for instance by insertional inactivation. It is also possible to delete the complete penicillin biosynthetic cluster comprising the genes encoding ACVS, IPNS and AT. In this way the production of the β-lactam compound of interest is possible without simultaneous production of the natural β-lactam. Insertional inactivation may thereby occur using a gene encoding a NRPS and/or a gene encoding an IPNS as described above. In host cells that contain multiple copies of β-lactam gene clusters, host cells wherein these clusters are spontaneously deleted may be selected. For instance, the deletion of β-lactam gene clusters is described in WO 2007/122249.

Another suitable host cell is a cell that is capable of synthesizing the precursor amino acids 4-hydroxyphenylglycine or phenylglycine. Heterologous expression of the genes of the biosynthetic pathway leading to 4-hydroxyphenylglycine or phenylglycine is disclosed in WO 2002/034921. The biosynthesis of 4-hydroxyphenylglycine or phenylglycine is achieved by withdrawing 4-hydroxyphenylpyruvate or phenylpyruvate, respectively, from the aromatic amino acid pathway, converting said components to 4-hydroxymandelic acid or mandelic acid, respectively, subsequently converting to 4-hydroxyphenylglyoxylate or phenylglyoxylate, respectively and finally converting to D-4-hydroxyphenylglycine or D-phenylglycine, respectively. Another suitable host cell is a cell that (over) expresses a 4′-phosphopantetheine transferase. 4′-Phosphopantetheine is an essential prosthetic group of amongst others acyl-carrier proteins of fatty acid synthases and polyketide synthases, and peptidyl carrier proteins of NRPS's. The free thiol moiety of 4′-phosphopantetheine serves to covalently bind the acyl reaction intermediates as thioesters during the multistep assembly of the monomeric precursors, typically acetyl, malonyl, and aminoacyl groups. The 4′-phosphopantetheine moiety is derived from coenzyme A and post translationally transferred onto an invariant serine side chain. This Mg²⁺-dependent conversion of the apoproteins to the holoproteins is catalyzed by the 4′-phosphopantetheine transferases. It is advantageous to (over)express a 4′-phosphopantetheine transferase with a broad substrate specificity. Such a 4′-phosphopantetheine transferase is for instance encoded by the gsp gene from Bacillus brevis as described by Borchert et al. (J. Bacteriol. (1994) 176, 2458-2462).

A host may suitably include one or more of the modifications as mentioned above. A preferred host is an organism capable of production under industrial conditions such as eukaryotes like Penicillium, Acremonium and Aspergillus examples of which are Penicillium chrysogenum, Acremonium chrysogenum, and Aspergillus nidulans.

### LEGEND TO THE FIGURES

FIGS. 1 to 4 depict the adenylation activity measurements with PPi Release assay for substrates L-phenylalanine (□), D-phenylalanine (▪), L-hydroxyphenylglycine () and D-hydroxyphenylglycine (▴) normalized for the incubation without substrate. X-axis: time (min); Y-axis: absorption (360 nm).

FIG. 1: For control protein TycA

FIG. 2: For StaA_M1_A

FIG. 3: For Veg8_M1_A

FIG. 4: For Veg8_M1_A and Tcp13

### EXAMPLES

### General Material and Methods

Molecular and Genetic Techniques

Standard genetic and molecular biology techniques are known in the art (e.g. Maniatis et al. “Molecular cloning: a laboratory manual” (1982) Cold Spring Harbor Laboratory, Cold Spring Harbor, N.Y.; Miller “Experiments in molecular genetics” (1972) Cold Spring Harbor Laboratory, Cold Spring Harbor, N.Y.; Sambrook and Russell “Molecular cloning: a laboratory manual” (3^{rd} edition)” (2001) Cold Spring Harbor Laboratory, Cold Spring Harbor Laboratory Press; Ausubel “Current protocols in molecular biology” (1987) Green Publishing and Wiley Interscience, New York).

Plasmids and Strains

pMAL-c5x was obtained from New England Biolabs Inc., pACYCtac has been described previously (M. Krämer “Untersuchungen zum Einfluss erhöhter Bereitstellung van Erythrose-4-Phosphat and Phosphoenolpyruvat auf den Kohlesrofffluss in den Aromatenbiosyntheseweg von Escherichia coil”, Berichte des Forschungszentrums Jülich, 3824, ISSN 0944-2952 (PhD Thesis, University of Dusseldorf). Escherichia coli strains Top10 (Invitrogen, Carlsbad, Calif., USA) or DH10b (Grant et al. (1990) Proc. Natl. Acad. Sci. USA (1990) 87, 4645-4649) were used for cloning and protein expression. Escherichia coli strain M15 pQE60-tycA pRep4 as described in Mootz, H. D. et al. (Proc. Natl. Acad. Sci. USA (2000) 97, 5848-53) and Mootz H. D. and Marahiel, M. A. (J Bacteriol. (1997) 179, 6843-6850) was kindly provided by Prof. M. Marahiel, Philipps University Marburg, Marburg, Germany.

Media

2xPY medium (16 g/l BD BBL™ Phytone™ Peptone, 10 g/l Yeast Extract, 5 g/l NaCl) was used for growth of Escherichia coli. Antibiotics (100 μg/ml ampicillin, or 50 μg/ml ampicillin together with 20 μg/ml chloramphenicol, or 100 μg/ml ampicillin together with 25 μg/ml neomycin depending on plasmids used) were supplemented to maintain plasmids. For induction of gene expression IPTG was used at 0.03-0.5 mM final concentration.

Identification of Plasmids

Plasmids carrying the different genes were identified by genetic, biochemical and/or phenotypic means generally known in the art, such as resistance of transformants to antibiotics, purification of plasmid DNA, restriction analysis of purified plasmid DNA or DNA sequence analysis.

Collection of Putative HPG Adenylation Domains from Existing NRPS Sequences in Uniprot/NCBI-ENV-PAT Databases

TABLE 1

| | | Module number | | | |
|---|---|---|---|---|---|
| | | in encoded | Module number | | |
| | | protein predicted | in predicted | SEQ ID NO: | |
| Uniprot | Encoded | to be specific | biosynthesis | adenylation | |
| code | protein | for HPG | cluster | domain | Organism |
| Q70AZ9 | Tcp9 | M1 | M1 | 1 | Actinoplanes |
| | | | | | teichomyceticus |
| Q7WZ66 | Dbv25 | M1 | M1 | 2 | Nonomuraea |
| | | | | | sp. ATCC 39727 |
| Q8KLL3 | StaA | M1 | M1 | 3 | Streptomyces |
| | | | | | toyocaensis |
| O52820 | CepB | M2 | M5 | 4 | Amycolatopsis |
| | (PCZA363.4) | | | | orientalis |
| Q939Z0 | BpsB | M2 | M5 | 5 | Amycolatopsis |
| | | | | | balhimycina |
| B7T1C1 | Veg8 | M1 | M4 | 6 | uncultured soil |
| | | | | | bacterium |
| Q70AZ7 | Tcp11 | M1 | M4 | 7 | Actinoplanes |
| | | | | | teichomyceticus |
| Q8KLL5 | StaC | M2 | M5 | 8 | Streptomyces |
| | | | | | toyocaensis |
| Q93N88 | ComB | M1 | M3 | 9 | Streptomyces |
| | | | | | lavendulae |
| Q939Z0 | BpsB | M1 | M4 | 26 | Amycolatopsis |
| | | | | | balhimycina |
| B7T1D2 | Teg7 | M1 | M4 | 27 | uncultured soil |
| | | | | | bacterium |

All proteins simultaneously containing the Pfam profiles characteristic for adenylation domains (Pfam identifier AMP-binding), Phosphopanthetheinyl-binding (Pfam identifier PP-binding) and condensation domains (Pfam identifier condensation) were collected from UniRef100 and NCBI env_nr and protein databases. These proteins are putative NRPS proteins. Putative NRPS protein sequences were selected from UniRef100 and NCBI env_nr and patent protein databases. Putative HPG adenylation domains were selected from NRPS's. In addition to predictions by the program NRPSpredictor (Rausch et al. (Nucleic Acids Res. (2005), 33, 5799-5808), the so-called Stachelhaus code (10 amino acids closest to the substrate bound in the active site (Stachelhaus et al. (Chem. & Biol. (1999) 6, 493-505)) was used, to predict the preferred amino acid bound by the adenylation domain of the identified NRPS Synthetase. Of the adenylation domains predicted to prefer 4-hydroxyphenylglycine, the following selection (Table 1) was made for biochemical characterization of adenylation specificity.

### Example 1

### Synthetic Design, Cloning, Expression, and Purification of NRPS Adenylation Domains which are Predicted as being Specific for L-hydroxyphenylglycine in Escherichia coli

Expression Constructs

Synthetic constructs codon optimized for Escherichia coli were designed for the adenylation domains with SEQ ID NO: 2-9, SEQ ID NO: 26, and SEQ ID NO: 27 as given above resulting in nucleotide SEQ ID NO: 10-17, SEQ ID NO: 28, and SEQ ID NO: 29, and ordered at DNA2.0. All were equipped with a C-terminal 6*His-tag for subsequent affinity chromatography (in appending a nucleotide sequence encoding the amino acid sequence GSRSHHHHHH) at the C terminus of the recombinant protein and flanked by restriction enzyme cloning sites NdeI/SbfI for subsequent cloning in the NdeI/SbfI sites of expression vector pMAL-c5x. The cloning of the synthetic DNA fragments in this vector results in the expression of a fusion protein of the respective A-domain with maltose binding protein at the N-terminus which allows high level of soluble protein expression by Escherichia coli. The final plasmids for overexpression of the adenylation domains constructed by cloning the NdeI/SfbI fragments taken from the synthetic constructs provided bt DNA2.0 into the NdeI/SbfI sites of expression vector pMAL-c5x were named pMAL-Dbv25_M1_A, pMAL-StaA_M1_A, pMAL-CepB_M2_A, pMAL-BpsB_M2_A, pMAL-Veg8_M1_A, pMAL-Tcp11_M1_A, pMAL-StaC_M2_A, pMAL-ComB_M1_A, pMAL-BpsB_M1_A, pMAL-Teg7_M1_A. In case of the construction of plasmid pMAL-StaA_M1_A, cloning by partial digestions of the synthetic construct SEQ ID NO: 11 with SbfI needed to be performed as the ordered fragment contained by mistake an additional SfbI site.

Protein Expression in Escherichia coli

Starter cultures of Escherichia coli harbouring plasmid pMAL-Dbv25_M1_A, or pMAL-StaA_M1_A, or pMAL-CepB_M2_A, or pMAL-BpsB_M2_A, or pMAL-Veg8_M1_A, or pMAL-Tcp11_M1_A, or pMAL-StaC_M2_A, or pMAL-ComB_M1_A, or pMAL-BpsB_M1_A, or pMAL-Teg7_M1_A were grown overnight at 37° C. in 3 ml 2*PY medium with 100 μg/ml ampicillin. The next day 100 ml 2*PY medium with 100 μg/ml ampicillin io in 0.5 l shake flask was inoculated with the preculture to an OD_{600 nm} of 0.015 and grown at 30° C. and 280 rpm. When an OD_{600 nm} of 0.4-0.6 was reached, the shake flask was cultured at 18° C. and 280 rpm for one hour. Following this temperature (pre-) adaptation, 3 μl of 1 M IPTG was added and the culture was grown at 18° C. and 220 rpm overnight.

Preparation of Cell Free Extracts and His-taq Purification:

Cells from 50 ml of the cultivations described in previous paragraph were harvested by centrifugation (5000 rpm, 10 minutes, 4° C.) and the pellets were re-suspended in 1 ml extraction buffer (50 mM Hepes pH 8.0, 5 mM DTT, 100 mM NaCl, 1× EDTA-free Complete protease inhibitor cocktail (Roche)). Cell lysis was obtained by sonification (9×10 sec. on/15 sec. off) keeping cells on ice during the procedure. To remove cell debris, the sonificated samples were centrifuged at 14.000 rpm for 15 min at 4° C. and the supernatants (cell free extracts) with the soluble proteins were transferred to fresh vials and kept on ice until further use. For purification of the His-tagged proteins TALON® Metal Affinity Resin was used according to the manufacturer's protocol (Clontech Laboratories, Inc. US; Protocol No. PT1320-1, Version No. PR6Z2142, page 30; VIII B Batch/Gravity-Flow Column Purification). Equilibration and washing of the column material was done with 50 mM Hepes pH8.0. Elution was done with 50 mM Hepes pH8.0+150 mM imidazole. 1 ml fractions were collected and kept on ice. The purified proteins are designated as Dbv25_M1_A, StaA_M1_A, CepB_M2_A, BpsB_M2_A, Veg8_M1_A, Tcp11_M1_A, StaC_M2_A, ComB_M1_A, BpsB_M1_A, or Teg7_M1_A.

Analyses Purified Proteins

By use of SDS-PAGE analysis (NuPAGE gels used according to manufacturers protocol) cell free extracts and the different elution fractions collected from the His-tag purification were analyzed for the presence of proteins and of correct size corresponding to the adenylation domains. For all adenylation domains over expressed, purification of a protein of the respective size was confirmed. The protein concentration of the different samples was determined using Coomassie Plus™ (Bradford) Assay Reagent (Thermo Scientific, PIERCE) according to the manufacturer's protocol.

### Example 2

### Expression and Purification of TycA Comprising Adenylation Domain Specific for Phenylalanine as Internal Control for Adenylation Activity Assay

Escherichia coli strain M15 pQE60-tycA pRep4 (see Plasmids and Strains) was used for overexpression and purification of TycA the first one-module-bearing peptide synthetase for synthesis of tyrocidine by Bacillus brevis. Expression and purification of TycA was performed as described in example 1, with the following variations. Antibiotics used in the medium were 100 μg/ml ampicillin and 25 μg/ml neomycin. Induction was done when the main culture was grown at 30° C. and 280 rpm to an OD₆₀₀ of 0.4-0.6 by addition of 50 μl of 1 M IPTG. After induction the cells were grown for additional 3 hours at 30° C. and 280 rpm before they were harvested. Preparation of cell lysates and protein purification was performed as described in Example 1.

### Example 3

### Synthetic Design and Cloning of MbtH-Like Proteins Tcp11, Tcp13 from Teicoplanin Cluster and VMbtH from Veg-Cluster

Three different MbtH-like proteins were chosen, two from the teicoplanin biosynthetic cluster annotated as tcp13 (SEQ ID NO: 18, GenBank: AJ605139 Genomic DNA; Translation: CAE53354.1) and tcp17 (SEQ ID NO: 19, GenBank: AJ605139 Genomic DNA; Translation: CAE53358.1) and one from the Veg biosynthetic clusters. The last one was named VMbtH, as it is not annotated in public databases yet and was identified by a search for homologous MbtH-like sequences in the Veg Cluster (SEQ ID NO: 20, GenBank: EU874252, nt 33826-34035, between veg9 and veg10). Target genes encoding the selected proteins were constructed synthetically (DNA2.0) resulting in nucleotide SEQ ID NO: 21-23 and ordered at DNA2.0. The genes encoding Tcp13 and Tcp17 were chosen as their wild type sequence, while the gene encoding VMbtH was codon optimized for expression in Escherichia coli. Each ORF was preceded by a consensus ribosomal binding site and flanked by restriction sites BamHI and SbfI for final cloning in expression plasmid pACYCtac. The final plasmids for overexpression of the MbtH-like proteins constructed by cloning the BamHI/SbfI fragments taken from the synthetic constructs provided bt DNA2.0 into the BamHI/SbfI sites of expression vector pACYCtac were named pACYCtac-Tcp13, pACYCtac-Tcp17 and pACYCtac-VMbtH.

### Example 4

### Synthetic Design and Cloning of MbtH-Like Proteins from Complestatine, Balhimycin and Teg-Cluster

Three additional MbtH-like proteins were chosen, one from the complestatine biosynthetic cluster annotated as hypothetical protein (SEQ ID NO: 30, GenBank: AF386507 Genomic DNA; Translation: AAK81828.1) and called CMbtH, one from the balhimycin biosynthetic cluster annotated as hypothetical protein and called BMbtH (SEQ ID NO: 31, GenBank: Y16952.3 Genomic DNA; Translation: CAC48363.1) and called BMbtH, and one from the Teg biosynthetic clusters. The last one is not annotated in public databases yet and was identified by a search for homologous MbtH-like sequences in the Teg Cluster (SEQ ID NO: 32, GenBank: EU874253, nt 32949-33158, between teg8 and teg9). It was called TMbtH. Target genes encoding the selected proteins were constructed synthetically (DNA2.0) resulting in nucleotide SEQ ID NO: 33-35 and ordered at DNA2.0 codon optimized for expression in Escherichia coli. All were equipped with a C-terminal 6*His-tag for possible affinity chromatography (in appending a nucleotide sequence encoding the amino acid sequence PGGHHHHHH) at the C terminus of the recombinant protein. Each ORF was preceded by a consensus ribosomal binding site and flanked by restriction sites BamHI and SbfI for final cloning in expression plasmid pACYCtac. The final plasmids for overexpression of the MbtH-like proteins constructed by cloning the BamHI/SbfI fragments taken from the synthetic constructs provided bt DNA2.0 into the BamHI/SbfI sites of expression vector pACYCtac were named pACYCtac-BMbtH, pACYCtac-CMbtH and pACYCtac-TMbtH.

### Example 5

### Co-Expression and Co-Purification of Adenylation Domains with MbtH Like Proteins

Escherichia coli strains harboring a pMAL plasmid for over expression of an adenylation domain as described in Example 1 and a pACYCtac plasmid for over expression of a MbtH-like protein as described in Example 3 and Example 4 were used for co-expression and co-purification of these two proteins. Expression and purification of an adenylation domain together with an MbtH-like protein was performed as described in Example 1, except that antibiotics used in the medium were 50 μg/ml ampicillin and 20 μg/ml chloramphenicol. By SDS page analysis of the elution fractions as described in Example 1, purification of two separate proteins was confirmed, one comprising the size of the respective adenylation domain, and another comprising the size of the MbtH-like protein. As the MbtH-like proteins Tcp13, Tcp17 and VMbtH are not equipped with a His-tag but nevertheless co-purified with the coexpressed adenylation domain, both proteins are tighly bound.

### Example 6

### Synthetic Design, Cloning, Expression, and Purification of an NRPS Adenylation-Thiolation Didomain with and without MbtH-Like Proteins

Expression Constructs

A synthetic construct was designed for the adenylation thiolation didomain comprising the wild type nucleotide sequence encoding SEQ ID NO: 1 together with its adjacent thiolation domain present in the Tcp9 encoding protein. This construct was equipped with a C-terminal 6*His-tag for subsequent affinity chromatography (in appending a nucleotide sequence encoding the amino acid sequence GSRSHHHHHH) at the C terminus of the recombinant protein and flanked by restriction enzyme cloning sites NdeI/SbfI for subsequent cloning in the NdeI/SbfI sites of expression vector pMAL-c5x. Cloning of the synthetic DNA fragment in this vector results in the expression of a fusion protein of the respective AT-didomain with maltose binding protein at the N-terminus which allows high level of soluble protein expression by Escherichia coli. The final plasmid for overexpression of the adenylation thiolation didomain constructed by cloning the NdeI/SfbI fragments taken from the synthetic constructs SEQ ID NO: 24 provided by DNA2.0 into the NdeI/SbfI sites of expression vector pMAL-c5x was named pMAL-Tcp9_M1_AT.

Protein expression and purification of the separate adenylation thiolation didomain was performed as described in Example 1, the purified protein was designated as Tcp9_M1_AT. Protein co-expression and co-purification of adenylation thiolation didomain together with an MbtH-like protein was performed as described in Example 5. By SDS page analysis of the elution fractions as described in sample 1, purification of either the separate adenylation thiolation didomain or two separate proteins was confirmed, one protein comprising the size of the respective adenylation thiolation didomain, and one protein comprising the size of the MbtH-like protein. As the MbtH-like proteins Tcp13, Tcp17 and VMbtH are not foreseen with a His-tag but nevertheless io purified together with the adenylation thiolation didomain, both proteins are tighly bound.

### Example 7

### Synthetic Design, Cloning, Expression, and Purification of an NRPS Adenylation-Thiolation-Epimerization Tridomain with and without MbtH-Like Proteins

Expression Constructs

A synthetic construct codon optimized for Escherichia coli was designed comprising the adenylation domain with SEQ ID NO: 6 and its adjacent thiolation domain and epimerization domain present in the Veg8 encoding protein. This construct was equipped with a C-terminal 6*His-tag for subsequent affinity chromatography (in appending a nucleotide sequence encoding the amino acid sequence GSRSHHHHHH) at the C terminus of the recombinant protein and flanked by restriction enzyme cloning sites NdeI/SbfI for subsequent cloning in the NdeI/SbfI sites of expression vector pMAL-c5x. Cloning of the synthetic DNA fragment in this vector results in the expression of a fusion protein of the respective ATE-tridomain with maltose binding protein at the N-terminus which allows high level of soluble protein expression by Escherichia coli. The final plasmid for overexpression of the adenylation thiolation didomain constructed by cloning the NdeI/SfbI fragments taken from the synthetic constructs SEQ ID NO: 25 provided by DNA2.0 into the NdeI/SbfI sites of expression vector pMAL-c5x was named pMAL-Veg8_M1_ATE.

Protein expression and purification of the separate adenylation thiolation epimerization tridomain was performed as described in Example 1, the purified protein was designated as Veg8_M1_ATE. Protein co-expression and co-purification of adenylation thiolation epimerization tridomain together with an MbtH-like protein was performed as described in Example 5.

By SDS page analysis of the elution fractions as described in sample 1, purification of either the separate adenylation thiolation didomain or two separate proteins was confirmed, one protein comprising the size of the respective adenylation thiolation didomain, and one protein comprising the size of the MbtH-like protein. As the MbtH-like proteins Tcp13, Tcp17 and VMbtH are not foreseen with a His-tag but nevertheless purified together with the adenylation thiolation epimerization tridomain, both proteins are tighly bound.

### Example 8

### Determination of Adenylation Activity for Putative L-hydroxyphenylglycine

Adenylation Domains, an Adenylation Thiolation Didomain and an Adenylation Thiolation Epimerization Tridomain by PPi Release Assay

To determine the adenylation activity of the adenylation domains, the Enzchek® pyrophosphate assay kit (Life Technologies) was used as described by Ehmann D. E. et al. (Proc Nat Acad Science (2000) 97, 2509-2514) with small modifications. The reactions were performed 96 wells UV/Vis transparent plates (BD Falcon). The reaction mixture comprises 50 mM HEPES pH 8.0, 10 mM MgCl2, 5 mM ATP, 75 mM DTT, 0.03 U Inorganic Pyrophosphatase (IP), 1 U Purine Nucleoside Phosphorylase (PNP) and 0.2 mM MESG in a volume of 70 μl. Next 20 μl (around 0.5-2 μM final concentration) of purified A(T) domain, with or without co-purification of the MbtH like helper protein was added and the reaction was pre-incubated for 15 minutes at RT to reduce contaminating Pi. Following the pre-incubation, 10 μl of a 10 mM or 1 mM solution of the appropriate amino acid depending on the performed specificity determination was added to initiate the adenylation reaction and the absorbance at 360 nm was measured using a TECAN I Control spectrophotometer. Absorbance measurements were made every 5 to 10 min over a period of up to 240 min. A reaction with addition of 10 μl MilliQ water instead was used to determine and subtract the background absorbance. As substrates the following amino acids were used: D- or L-phenylalanine, D- or L-hydroxyphenylglycine, D- or L-phenylglycine, L-tryptophan, L-valine, L-cysteine, and L-leucine.

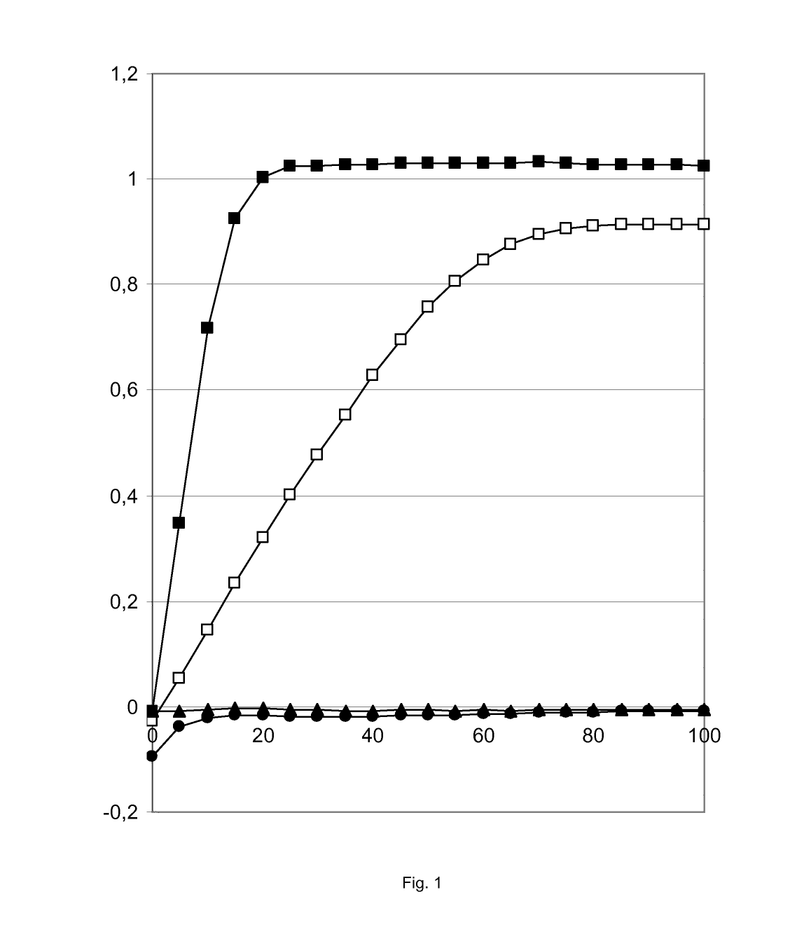

FIG. 1 shows a graph of the absorption measurements of the PPi release assay with the control protein TycA. While L- and D-phenylalanine are accepted as substrate, no adenylation activity is measured for L- and D-hydroxyphenylglycine. Beside L- and D-phenylalanine, also L-tryptophan, L-valine and L-leucine (data not shown) have been shown to be similarly recognized and adenylated by TycA while no adenylation activity was measured for L-cysteine (data not shown) which is in agreement with the findings of Villiers and Hollfelder (ChemBioChem (2009) 10, 671 -682).

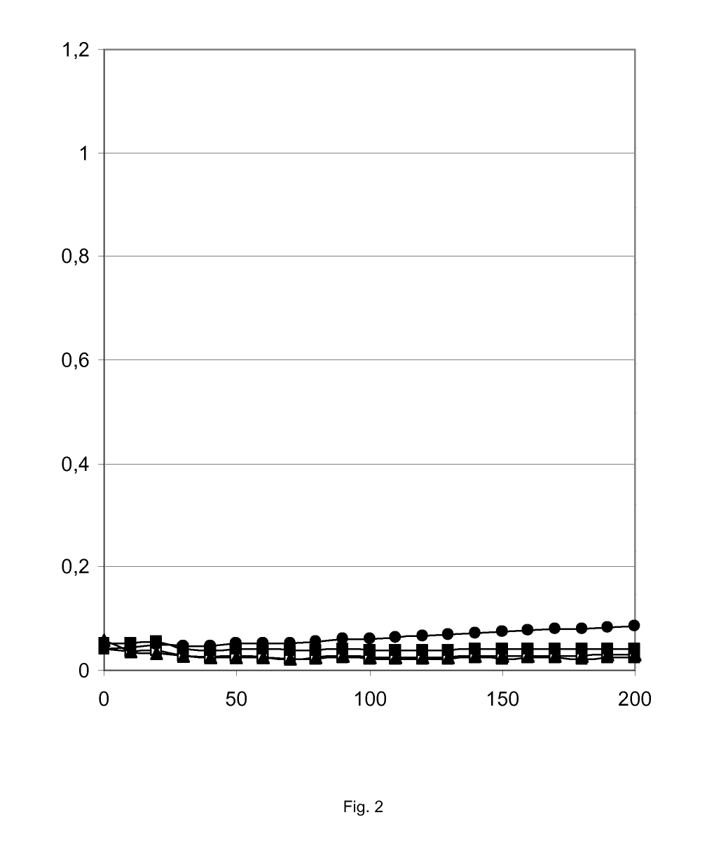

FIG. 2 shows a graph for the absorption measurements of the PPi release assay with the single adenylation domain derived from StaA_M1_A. No adenylation activity is determined for the amino acids L- or D-hydroxyphenylglycine, nor L- or D-phenylalanine. The graphs for the adenylation domains Dbv25_M1_A, CepB_M2_A, BpsB_M2_A, Tcp11_M1_A, StaC_M2_A, ComB_M1_A, BpsB_M1_A, Teg7_M1_A, or the adenylation thiolation didomain of Tcp9_M1_AT gave the same results (data not shown). No adenylation activity could be confirmed for L- or D-hydroxyphenylglycine.

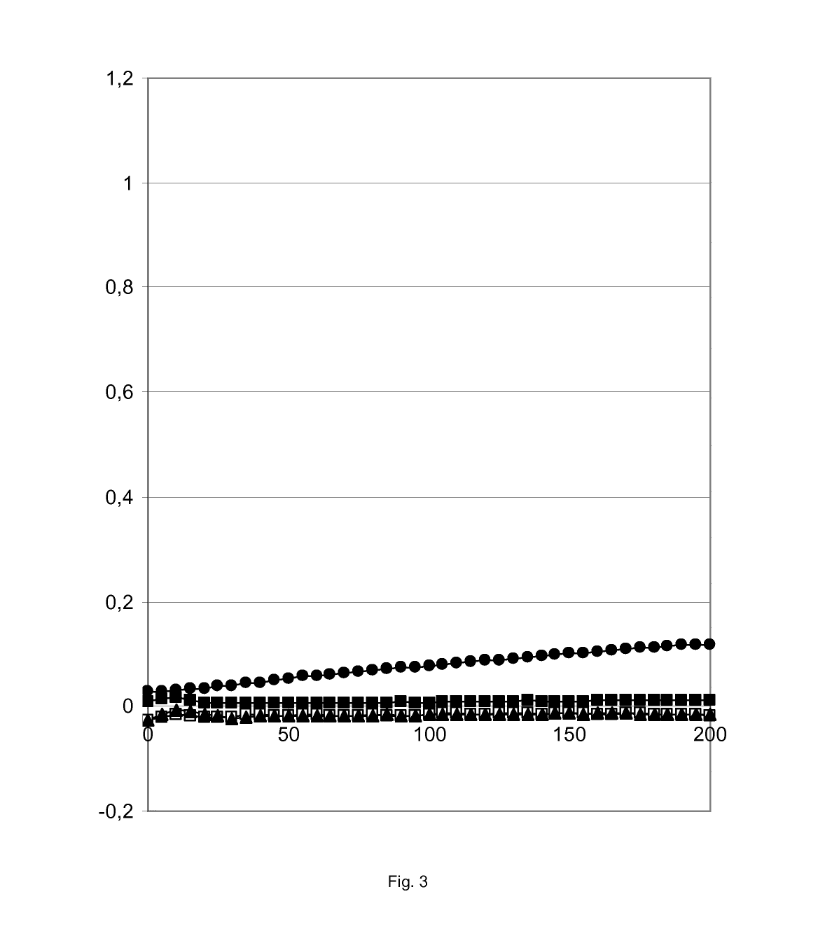

FIG. 3 shows a graph for the absorption measurements of the PPi release assay with the single adenylation domain derived from VegA_M1_A. A very minor adenylation activity is determined for the amino acids L-hydroxyphenylglycine, while no activity was determined for D-hydroxyphenylglycine, D- and L-phenylalanine.

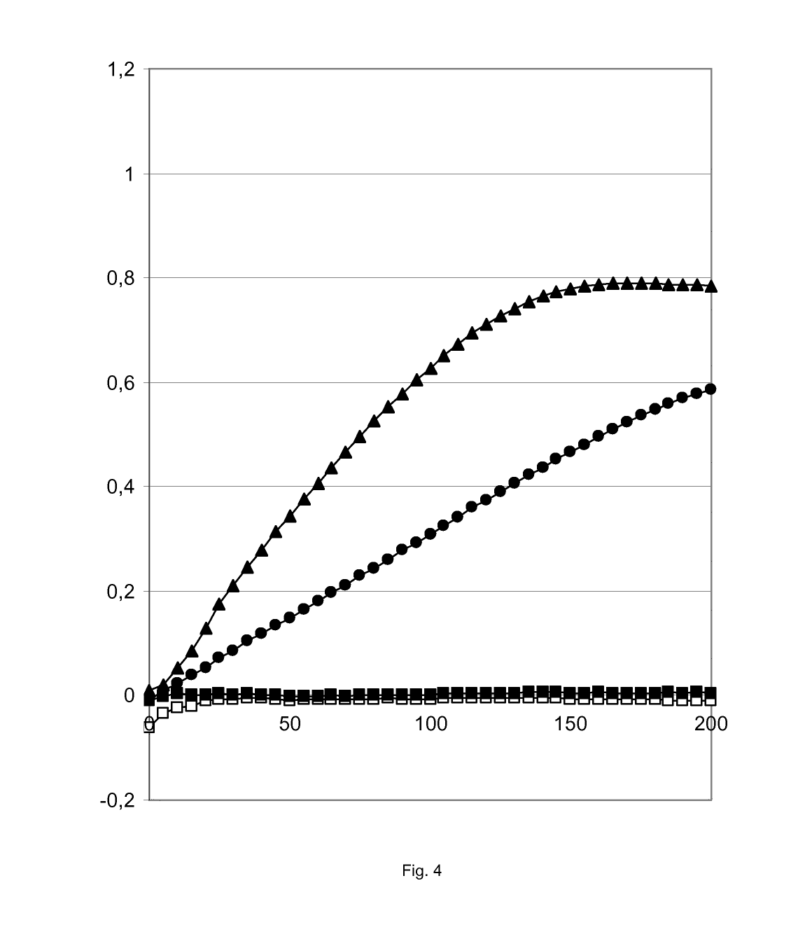

FIG. 4 shows a graph for the absorption measurements of the PPi release assay with the single adenylation domain derived from VegA_M1_A co-purified with the MbtH-like protein Tcp13. A clear adenylation activity is determined for the amino acids L- and D-hydroxyphenylglycine, while no activity is determined for L- or D-phenylalanine. The graphs for the adenylation activity determinations of CepB_M2_A, BpsB_M2_A, Tcp11_M1_A, StaC_M2_A, or the adenylation thiolation didomain of Tcp9_M1_AT or the adenylation thiolation epimerisation tridomain of Veg8_M1_ATE all co-purified with the MbtH-like protein Tcp13 show the same results (data shown in Table 3). The graphs for the adenylation activity determinations of StaA_M1_A, and Dbv25_M1_A both co-purified with the MbtH-like protein VMbtH show the same results (data shown in Table 3).

Table 2 gives an overview on the adenylation activity determinations performed for single adenylation domains Tcp11_M1_A and VegA_M1_A, the adenylation thiolation didomain of Tcp9_M1_AT or the adenylation thiolation epimerisation tridomain of Veg8_M1_ATE all co-purified with the MbtH-like protein Tcp13, or Tcp17 or VMbtH given in amount of PPi formed per minute and mM of protein. In the adenylation activity determinations of ComB_M1_A, BpsB_M1_A, Teg7_M1_A all co-purified with the MbtH-like protein Tcp13, or Tcp17 or VMbtH no adenylation activity with D- or L-hydroxyphenylglycine, D- or L-phenylglycine D- (data shown in Table 3) or L-phenylalanine is determined. The adenylation activity determination of ComB_M1_A co-purified with the MbtH-like protein CMbtH derived from the same biosynthetic cluster as the A-domain confirmed its activity with L- or D-hydroxyphenylglycine; the adenylation activity determination of BpsB_M1_A co-purified with the MbtH-like protein BMbtH derived from the same biosynthetic cluster as the A-domain confirmed its activity with L-hydroxyphenylglycine, and the same specificity was determined in the adenylation activity determination of Teg7_M1_A co-purified with the MbtH-like protein TMbtH.

Table 3 gives a general overview on the adenylation activity determinations performed for the different amino acid substrates and the different combinations of either single adenylation domains, or the adenylation thiolation didomain of Tcp9_M1_AT or the adenylation thiolation epimerisation tridomain of Veg8_M1_ATE with the co-purified MbtH-like proteins Tcp13, or Tcp17 or VMbtH or CMbtH or BMbtH or TMbtH and the relative adenylation activities determined.

TABLE 2
Adenylation activity determinations by PPi release assay of
Tcp11_M1_A, Veg8M1_A, Tcp9_M1_AT and Veg8_M1_ATE in
combination with MbtH like helper proteins Tcp13, Tcp17 or VMbtH.

| | | Formed PPi | | |
|---|---|---|---|---|
| | | (mM/min/mM enzyme) | | |
| Purified protein | Substrate | Tcp 13 | Tcp 17 | VMbtH |
| Tcp11_M1_A | D-HPG 1 mM | 0.66 | 0.63 | 0.86 |
| | D-HPG 0.1 mM | 0.08 | 0.11 | 0 |
| | L-HPG 1 mM | 1.03 | 1.04 | 1.54 |
| | L-HPG 0.1 mM | 0.80 | 0.95 | 1.38 |
| | D-PG 1 mM | 0 | 0.04 | 0 |
| | L-PG 1 mM | 0.17 | 0.23 | 0.09 |
| Veg8_M1_A | D-HPG 1 mM | 0.92 | 1.03 | 1.39 |
| | D-HPG 0.1 mM | 0.14 | 0.17 | 0.18 |
| | L-HPG 1 mM | 0.59 | 0.64 | 0.61 |
| | L-HPG 0.1 mM | 0.56 | 0.70 | 0.61 |
| | D-PG 1 mM | 0.01 | 0.02 | 0.02 |
| | L-PG 1 mM | 0.17 | 0.14 | 0.20 |
| Tcp9_M1_AT | D-HPG 1 mM | 5.28 | 4.63 | 8.44 |
| | D-HPG 0.1 mM | 2.07 | 1.71 | 3.72 |
| | L-HPG 1 mM | 1.16 | 1.34 | 1.40 |
| | L-HPG 0.1 mM | 1.18 | 1.20 | 1.23 |
| | D-PG 1 mM | 0.05 | 0.05 | 0.07 |
| | L-PG 1 mM | 1.32 | 1.44 | 2.32 |
| Veg8_M1_ATE | D-HPG 1 mM | 0.72 | 0.62 | 1.42 |
| | D-HPG 0.1 mM | 0.12 | 0.11 | 0.27 |
| | L-HPG 1 mM | 0.57 | 0.52 | 0.88 |
| | L-HPG 0.1 mM | 0.54 | 0.48 | 0.84 |
| | D-PG 1 mM | 0.01 | 0.01 | 0.02 |
| | L-PG 1 mM | 0.15 | 0.12 | 0.27 |

TABLE 3

| Adenylation- | MbtH-like | Substrates | | | | |
|---|---|---|---|---|---|---|
| domain | protein | L-HPG | D-HPG | L-PG | D-PG | L-Phe |
| StaA_M1_A | VMbtH | +++ | +++ | +++ | − | − |
| Dbv25_M1_A | VMbtH | +++ | +++ | +++ | − | − |
| StaC_M2_A | Tcp13 | ++ | ++ | − | − | − |
| Tcp11_M1_4 | Tcp13/ | +++ | +++ | +++ | − | − |
| | Tcp17/ | | | | | |
| | VMbtH | | | | | |
| Veg8_M1_A | Tcp13/ | +++ | +++ | +++ | − | − |
| | Tcp17/ | | | | | |
| | VMbtH | | | | | |
| BpsB_M2_A | Tcp13 | +++ | +++ | +++ | − | − |
| CepB_M2_A | Tcp13 | +++ | +++ | +++ | − | − |
| Tcp9_M1_AT | Tcp13/ | +++ | +++ | +++ | − | − |
| | Tcp17/ | | | | | |
| | VMbtH | | | | | |
| Veg8_M1_ATE | Tcp13/ | +++ | +++ | +++ | − | − |
| | Tcp17/ | | | | | |
| | VMbtH | | | | | |
| ComB_M1_A | Tcp13/ | − | − | − | − | − |
| | Tcp17/ | | | | | |
| | VMbtH | | | | | |
| BpsB_M1_A | Tcp13/ | − | − | − | − | − |
| | Tcp17/ | | | | | |
| | VMbtH | | | | | |
| Teg7_M1_A | Tcp13/ | − | − | − | − | − |
| | Tcp17/ | | | | | |
| | VMbtH | | | | | |
| ComB_M1_A | CMbtH | +++ | + | − | − | − |
| BpsB_M1_A | BMbtH | ++ | − | − | − | − |
| Teg7_M1_A | TMbtH | +++ | − | − | − | − |

1. A method for the preparation of an N-α-amino-4-hydroxyphenylacetyl or an N-α-am inophenylacetyl β-lactam antibiotic comprising the steps of:
(a) contacting the amino acids 4-hydroxyphenylglycine or phenylglycine, cysteine and valine with a non-ribosomal peptide synthetase to give a tripeptide 4-hydroxyphenylglycyl-cysteinyl-valine or a tripeptide phenylglycyl-cysteinyl-valine, respectively;
(b) contacting the tripeptide obtained in step (a) with an isopenicillin N synthase,
characterized in that an MbtH-like protein is present.

2. Method according to claim 1 wherein said MbtH-like protein has SEQ ID NO: 18, SEQ ID NO: 19, SEQ ID NO: 20, SEQ ID NO: 30, SEQ ID NO: 31 or SEQ ID NO: 32 or a sequence that is at least 50% homologous to SEQ ID NO: 18, SEQ ID NO: 19 or SEQ ID NO: 20, SEQ ID NO: 30, SEQ ID NO: 31 or SEQ ID NO: 32.

3. Method according to claim 1 wherein said MbtH-like protein has the amino acid code NXEXQXSXWP-X₅-PXGW-X₁₃-L-X₇-WTDXRP.

4. Method according to claim 1 wherein said non-ribosomal peptide synthetase comprises a first module M1 specific for 4-hydroxyphenylglycine and/or phenylglycine, a second module M2 specific for cysteine and a third module M3 specific for valine.

5. Method according to claim 1 which is carried out in a eukaryotic microorganism.

6. Method according to claim 5 wherein said eukaryotic microorganism is Penicillium.

7. Method according to claim 4 wherein said β-lactam antibiotic is an N-α-aminophenylacetyl β-lactam antibiotic and said first module M1 comprises an adenylation domain chosen from the list consisting of SEQ ID NO: 1, SEQ ID NO: 2, SEQ ID NO: 3, SEQ ID NO: 4, SEQ ID NO: 5, SEQ ID NO: 6, SEQ ID NO: 7 and a sequence that is at least 50% homologous to SEQ ID NO: 1, SEQ ID NO: 2, SEQ ID NO: 3, SEQ ID NO: 4, SEQ ID NO: 5, SEQ ID NO: 6 or SEQ ID NO: 7.

8. A eukaryotic host cell comprising a non-ribosomal peptide synthetase, an isopenicillin N synthase and a polynucleotide allowing the expression of an MbtH-like protein.

9. Host cell according to claim 8 wherein said MbtH-like protein has SEQ ID NO: 18, SEQ ID NO: 19, SEQ ID NO: 20, SEQ ID NO: 30, SEQ ID NO: 31 or SEQ ID NO: 32 or a sequence that is at least 50% homologous to SEQ ID NO: 18, SEQ ID NO: 19 or SEQ ID NO: 20, SEQ ID NO: 30, SEQ ID NO: 31 or SEQ ID NO: 32.

10. Host cell according to claim 8 wherein said MbtH-like protein has the amino acid code NXEXQXSXWP-X₅-PXGW-X₁₃-L-X₇-WTDXRP.

11. Host cell according to claim 8 which is Penicillium chrysogenum, Acremonium chrysogenum or Aspergillus nidulans.
